(19) United States
(12) Patent Application Publication
(10) Pub. No.: US 2020/0046450 A1
(43) Pub. Date: Feb. 13, 2020

(54) MAGNETIC RESONANCE IMAGING COMPATIBLE SYSTEM FOR IMPARTING MOTION

(71) Applicant: THE REGENTS OF THE UNIVERSITY OF CALIFORNIA, Oakland, CA (US)

(72) Inventors: Tsu-Chin TSAO, Los Angeles, CA (US); James M. SIMONELLI, Los Angeles, CA (US); Holden H. WU, Los Angeles, CA (US); Samantha MIKAIEL, Los Angeles, CA (US); Yu-Hsiu LEE, Los Angeles, CA (US); Cheng-Wei CHEN, Los Angeles, CA (US); Kyung Hyun SUNG, Los Angeles, CA (US); David S. LU, Los Angeles, CA (US)

(21) Appl. No.: 16/486,455

(22) PCT Filed: Feb. 20, 2018
(86) PCT No.: PCT/US2018/018850

§ 371 Date: Aug. 15, 2019

Related U.S. Application Data
(60) Provisional application No. 62/461,522, filed on Feb. 21, 2017.

(51) Int. Cl. A61B 90/11; A61B 5/055; A61B 5/00; F15B 7/00

(52) U.S. Cl. CPC A61B 90/11; A61B 5/055; A61B 2090/374; F15B 7/00; A61B 5/0036

(57) ABSTRACT

A system for imparting motion of an object includes: (1) at least one first hydrostatic actuator; (2) a hydraulic transmission conduit; and (3) at least one second hydrostatic actuator. The first hydrostatic actuator is connected to the second hydrostatic actuator via the hydraulic transmission conduit, such that an input displacement applied to the first hydrostatic actuator is transmitted via the hydraulic transmission conduit to the second hydrostatic actuator to impart motion of the object.

### CROSS-REFERENCE TO RELATED APPLICATION

This application claims the benefit of U.S. Provisional Application No. 62/461,522, filed Feb. 21, 2017, the contents of which are incorporated herein by reference in their entirety.

### TECHNICAL FIELD

This disclosure generally relates to a system for imparting motion.

### BACKGROUND

Magnetic Resonance (MR) imaging is desirable for guiding minimally invasive interventions. However, a constraint is that a narrow gantry space of an MR scanner restricts access to a patient by a physician. A remotely-controlled system would be desirable to overcome the challenge of physical space restrictions in the MR scanner. Unfortunately, due to strong electromagnetic fields in an MR imaging environment, actuation mechanisms that operate based on electromagnetic interactions or include magnetic materials are generally not acceptable.

It is against this background that a need arose to develop the embodiments described herein.

### SUMMARY

In some embodiments, a system for imparting motion of an object includes: (1) a first hydrostatic actuator; (2) a hydraulic transmission conduit; and (3) a second hydrostatic actuator. The system also optionally includes (4) a controller. The first hydrostatic actuator is connected to the second hydrostatic actuator via the hydraulic transmission conduit, such that an input displacement applied to the first hydrostatic actuator is transmitted via the hydraulic transmission conduit to the second hydrostatic actuator to impart motion of the object. The input displacement can be applied manually or through the optional controller.

In some embodiments, the system of the foregoing embodiments is operated by a method that includes: (1) placing the second hydrostatic actuator within an MR scanner bore; and (2) imparting motion of the object, via the second hydrostatic actuator, while the object is within the MR scanner bore. The motion of the object can be specified, via on-line or off-line measurement control applied to the actuators.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1. Schematic of a system for body motion emulation.

FIG. 2. Cross-section of a double-acting diaphragm actuator.

FIG. 3. Image of a fabricated double-acting diaphragm actuator.

FIG. 4. System in a needle targeting experiment.

FIG. 5. Schematic of a master unit.

FIG. 6. Tracking experimental setup.

FIG. 7. Feedforward control scheme.

FIG. 8. Iterative Learning Control (ILC) scheme.

FIG. 9. Feedforward inversion tracking results.

FIG. 10. Inversion-based ILC tracking results.

FIG. 11. ILC convergence plot.

FIG. 12. (a) Schematic and (b) image of a rolling diaphragm hydrostatic actuator. (c) Master-slave actuator pair connected by water-filled fluid lines and stabilized with an adjustable positioning frame. A needle is attached to the slave actuator for targeted needle placement.

FIG. 13. Diagram (left) and image (right) of an interventional Magnetic Resonance (MR) imaging suite.

FIG. 14. (a) Axial MR image of a targeting plate in a phantom (outline). (b) Images of the platform with an outer layer. (c) Relative positioning of an actuator system, motion platform, and targeting phantom filled with gelatin for experiments.

FIG. 15. Measurement of a contrast bead at baseline (left) and about 20-mm insertion (right).

FIG. 16. (a) Transverse and (b) coronal planning MR imaging scans with a target specified (X). The dashed line illustrates a slice position selected for a coronal plane. Zoomed-in views (square) illustrate the determination of a needle (signal void) to target error (NTE) in X, Y and Z.

FIG. 17. Flow chart of Step-and-Shoot and Actuator-Assisted methods for targeted needle placement experiments. The dotted line signifies a breath-hold for dynamic phantom experiments.

FIG. 18. 3D Gradient Echo (GRE) images of a sphere phantom with corresponding signal-to-noise ratio (SNR) values (a) without and (b) with an actuator. 3D GRE images of an American College of Radiology (ACR) phantom with corresponding Horizontal (H) and Vertical (V) grid line lengths and Angle (c) without and (d) with the actuator.

FIG. 19. Insertion and retraction of a master actuator versus a measured displacement for a slave actuator.

FIG. 20. Box and whisker plots for NTE of (a) Static and (b) Dynamic targeting experiments.

FIG. 21. Box and whisker plots for time of each step of (a) Static and (b) Dynamic targeting experiments.

FIG. 22. MR robotics system schematic.

FIG. 23. Motion scaling experimental results.

FIG. 24. Frequency crossover experimental results.

FIG. 25. Virtual Wall experimental results.

FIG. 26. Example recorded breathing trajectory.

FIG. 27. Adaptive feedforward controller.

FIG. 28. Adaptive feedback controller.

FIG. 29. Adaptive feedback/feedforward controller.

FIG. 30. Schematic of a controller.

### DETAILED DESCRIPTION

Magnetic Resonance Imaging Compatible System for Body Motion Emulation

Overview:

In some embodiments, this disclosure presents a system that physically emulates respiratory motion for phantoms under Magnetic Resonance (MR) imaging. The system is designed to be MR-compatible—with no ferrous materials or electromagnetic actuators present in an exam room—and uses hydrostatic actuators to control motion of the phantom. Signal-to-noise ratio (SNR) and imaging distortion tests have been performed to show MR-compatibility of the system. Several control strategies are also presented to achieve tracking of pre-recorded respiratory motion profiles obtained from a human subject.

Introduction:

Minimally-invasive interventions guided by MR are a very promising avenue in the field of interventional oncology. Physicians can use a combination of ultrasound and Computed Tomography (CT) imaging to target cancerous lesions on internal organs, such as the liver. Ultrasound provides “real-time” imaging, but suffers from poor image quality; CT provides high-resolution imaging, but due to radiation exposure, generally cannot be performed continuously. MR combines the best qualities of these imaging options—it can provide reasonably high-resolution imaging continuously because there are no known health risks associated with magnetic fields.

It can be beneficial for physicians to refine their techniques and for the physicians to be provided with better imaging. To do this, one can simulate the conditions of a patient using phantoms. Phantoms simulating various body areas can be used, including “gel phantoms” formed of gelatin or agar to simulate flesh. However, a gel phantom is stationary. While it is possible to keep a patient virtually stationary by holding his or her breath, there is a time constraint to this technique, and it is not repeatable from trial to trial, or from patient to patient. When breathing normally, data collected from human subjects shows that internal structures can move in excess of 50 millimeters over the course of a breath. This presents issues for both a physician and an MR scientist—the physician would have to hit a moving target, and the MR scientist would have to contend with artifacts introduced into an image by the breathing motion. Simulation of the respiratory motion can be beneficial for the development of MR imaging and interventional techniques.

A robotic system can provide several advantages over a traditional animal study: a robot is repeatable, so that an MR scientist can isolate changes in the motion as a variable and focus exclusively on the scan parameters; a robot is configurable so a physician can simulate patient-to-patient variability when desired, but also have the freedom to exploit the repeatability of the system to hone the technique; and a robot is convenient—a robot specifies no additional personnel like a live-animal study, without ethical concerns or need for oversight.

However, an MR environment itself can pose challenges to the implementation of such a system. Due to the high magnetic fields present at all times in a scanner bore—between about 1.5 Tesla (T) to about 3 T—any ferromagnetic components, or electromagnetic actuators and sensors are prohibited due to safety concerns. In addition to the safety issues, these components also can introduce artifacts that degrade an imaging obtained from the scanner. These concerns alone impede the use of various actuation/sensing modalities and materials, complicating the design of a robotic system. To resolve this, one approach presents a remotely actuated MR-compatible motion phantom, using remote motor actuation of a stepper motor as a direct drive for open-loop control precision and repeatability. However, there are several disadvantages with this approach. For example, the stepper motor should be well shielded and secured at a safe distance. Rigid connection for motion transmission can take up inflexible space and make the demand in component precision stricter. Further, the phantom motion precision can also be compromised since the dynamics from a motor shaft input to an actual phantom motion output may not be taken into account.

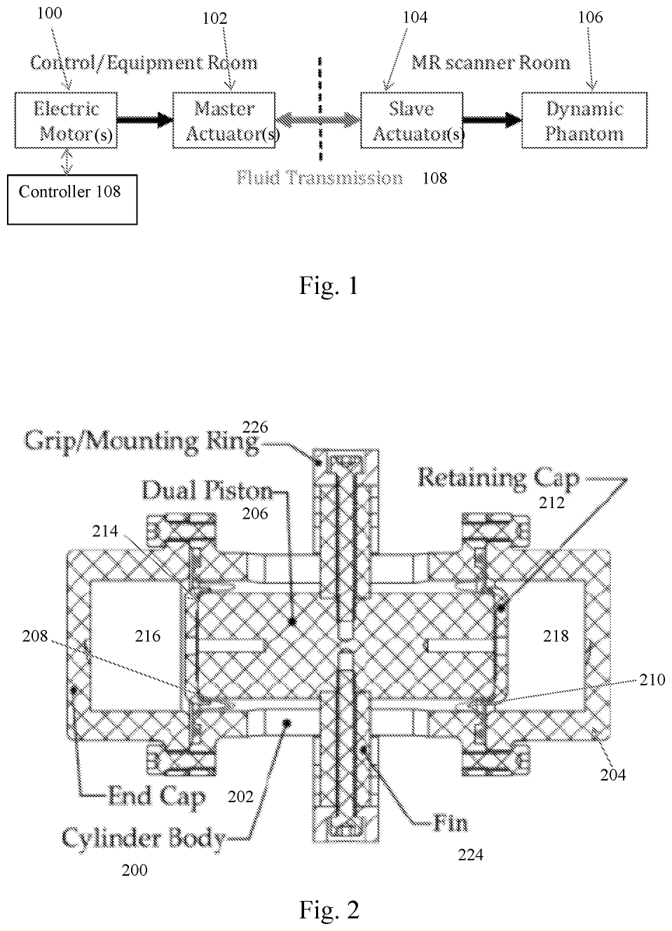

In some embodiments, the system discussed herein is specifically designed to be MR-compatible—that is, no ferrous materials or electromagnetic actuators are present in an exam room. As shown in FIG. 1, the system incorporates use of a hydraulic fluid transmission of force and displacement, via a hydraulic or fluid transmission line or conduit 108, from a master actuator 102, which is actuated by an electric motor 100 and both of which are placed outside the exam room, to a slave actuator 104 placed in a scanner bore and which actuates motion of a phantom 106 (or another object). Because of this indirect transmission, dynamics can arise from both hydraulics and the mechanisms coupling them to input and output stages. More generally, the system can include one master actuator 102 or multiple master actuators 102, which may have the same or different sizes (e.g., in terms of piston cross-sectional areas), can include one electric motor 100 or multiple electric motors 100 connected to at least a subset of the multiple master actuators 102, and can include one slave actuator 104 or multiple slave actuators 104, which may have the same or different sizes (e.g., in terms of piston cross-sectional areas). The system also includes a controller 108, which is connected to and directs operation of the electric motor 100. The system can include three control schemes: an inner-loop proportional-integral (PI) on the master side, a data-based feedforward dynamic inversion, and an Iterative Learning Control (ILC) scheme using the aforementioned dynamic inversion as its learning filter. In particular, this disclosure presents the design and verification of the MR-compatible system to emulate respiratory motion, comparison of three control strategies to track respiratory motion recorded from a human subject, and results from a modified system to increase stroke.

More generally and according to some embodiments, the system physically emulates respiratory or other motion of phantoms under MR imaging. This system embodies two aspects. The first is the design and implementation of MR compatible hydrostatic actuators in the system. MR compatibility of the system is verified through both SNR and imaging distortion tests. The second aspect includes control of an actuator to generate precise mechanical motion without requiring use of real-time displacement feedback sensors in an MR chamber. The control technique can apply iterative learning control to precisely track a desired trajectory with MR-incompatible displacement feedback sensors. The obtained actuator signals are then applied as feedforward control signal with the sensors removed to render the system MR-compatible and to reproduce precise trajectory in MR imaging under motion or MR imaging guided interventions. The system presented herein mitigates against imaging artifacts, and provides flexibility for placement anywhere inside a magnet without requiring rigid or stiff construction.

System Design:

Actuators

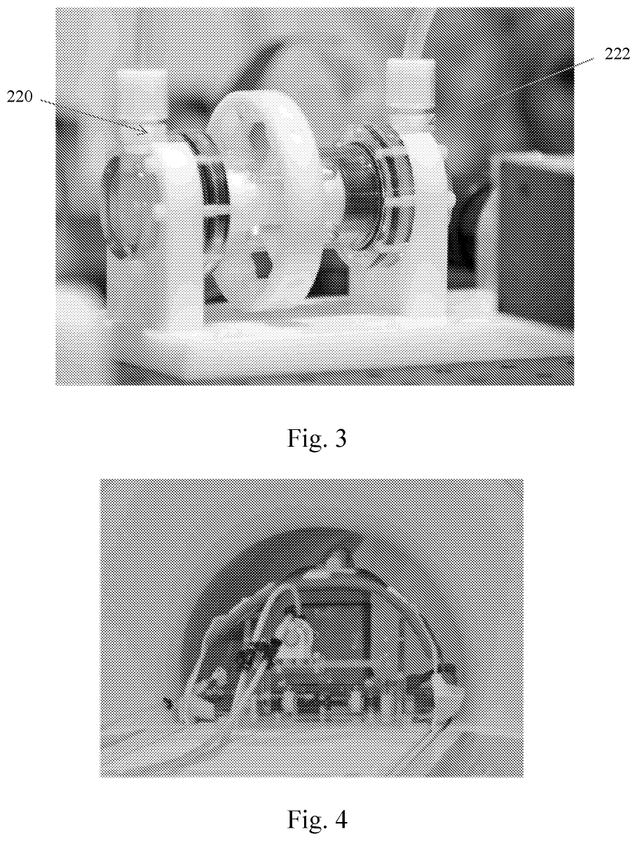

The following discussion presents a double-acting, hydrostatic linear actuator with a stroke of about 25 millimeters. A cross-section of the actuator is shown in FIG. 2 and an image of the fabricated actuator is shown in FIG. 3. One design consideration for the actuator includes the exclusion of ferromagnetic materials, as well as metals. In some embodiments, the actuator has a material composition that is primarily or entirely non-metallic and non-ferromagnetic, such as one that is primarily or entirely polymeric and including one or more plastics. While the MR environment dictates this consideration, the implementation of a pseudo-open-loop system specifies the inclusion of actuators and mechanisms with high stiffness. Finally, the confines of the MR bore place size constraints, so a compact implementation is desired. A rolling diaphragm piston design is implemented due to its desirable properties for the MR application. As shown in FIG. 2, the actuator includes a hollow cylinder body 200 having two ends, a pair of end caps 202 and 204 affixed to respective ends of the cylinder body 200, and a dual piston 206 moveably disposed within an interior defined by the cylinder body 200 and the end caps 202 and 204. The cylinder body 200 together with the end caps 202 and 204 also can be referred to as an actuator body. A pair of diaphragms 208 and 210 formed of a flexible or stretchable material are included, and each diaphragm 208 and 210 is affixed to and extends between a respective end cap 202 or 204 and a respective end of the dual piston 206 (and is affixed thereto via a retaining cap 212 or 214). A pair of chambers 216 and 218 are defined by the diaphragms 208 and 210 and the end caps 202 and 204. The rolling diaphragm design offers several advantages, such as: a hermetic seal of a cylinder volume between the diaphragms 208 and 210, regardless of the position or movement of the piston 206; and low friction. Each piston head is designed with a fluid port 220 or 222 (see FIG. 3) as a sole or main opening in the chamber 216 or 218, and uses a bolt-through-flange mechanism to seal a stationary end of the diaphragm 208 or 210. This allows ready re-assembly of the actuator upon changing diaphragms or other components. Another option for the double-acting design includes two pistons, rigidly connected together. In the presented design, the two pistons are a single component in the form of the dual piston 206, with four fins 224 as extension members that extend outwardly from the piston 206 and guide movement of the piston 206, and extend through slots in the cylinder body 200 and are affixed to a mounting member 226 in the form of a ring to bring the movement out of the confines of the cylinder body 200 and accept attachments. These four fins 224 also act as linear bearings to guard against torquing the diaphragms 208 and 210, although the fins 224 are designed with large clearance to allow the piston 206 to “float” with the pair of diaphragms 208 and 210 acting as bearings, so an external linear bearing is specified for applications that have reduced tolerance to angular play in the mechanism. In some implementations, during normal operation, none of the four fins 224 directly contact the cylinder body 200, so they add no friction.

In some implementations, a plastic (e.g., polyoxymethylene) can be used as a material for the piston 206, the fins 224, compression fittings (e.g., retaining caps 212 and 214), and the ring 226 for its excellent machinability and sliding friction. The cylinder body 200 and the end caps 202 and 204 (cylinder heads) can be formed of acrylic, both for its sliding friction properties (for slots in the cylinder body 200) and for its transparency.

Input and Output Coupling

Slave Side—Phantom Platform.

Two variations of a Phantom Platform are discussed herein: the majority of the results are from an implementation that features an about 1:1 transmission of motion from master to motion platform. Results are also presented below from another implementation that adds a mechanical amplification scheme to increase the transmission to greater than about 1:1, namely about 2:1. Both versions can use a common design, with the amplification scheme is added as a bolt-on system.

Plastic components are selected to avoid the usage of metals, and no ferromagnetic materials are used. A base plate of a stage is designed to mate with a scanner bed through a set of slots that accept the same straps used to secure a body coil, allowing the stage to be mounted anywhere along the bed. An additional set of slots, rotated about 90 degrees, allows the stage to be mounted with its axis of motion either parallel to the axis of the scanner or transverse. The slave actuator is affixed to the base plate and attached to a linear guide to serve as an external bearing. In the about 1:1 design, the moving stage plate is directly attached to this guide, while in the about 2:1 design, a pulley is used, and the stage is connected to the actuator through a cable.

The moving stage plate slides on a pair of carbon fiber rails through four linear guides, and features hard attachment points for a gel phantom. A custom gel phantom was formed to mount to the stage, and features an adjustable target plate that can sink into various depths into the gel phantom. This provides a repeatable set of targets for imaging or interventional assessments. FIG. 4 shows the system simulating breathing motion in a targeting experiment.

To increase the stroke of the actuator, a pulley mechanism is designed to mechanically amplify the stroke of the actuator by a factor of about two with reduced backlash and friction. This involves fixing one end of the cable to the base plate, and the other to the moving stage plate. The cable passes around a pulley held in the linear guide attached to the actuator, and around two idlers to redirect the tension force to the horizontal center of the moving plate, as to avoid creating unnecessary torques and loads on the linear guide. Since a pulley mechanism has a directional preference, a return mechanism is added to provide force in the other direction. An elastic cord, anchored to the base plate, is used as a return spring. The spring constant and preload distance are chosen so to balance the competing goals of keeping tension in the cable, and not overtaxing the master-side drive motor's torque specifications. FIG. 6 shows the bolt-on amplification mechanism.

Master Side.

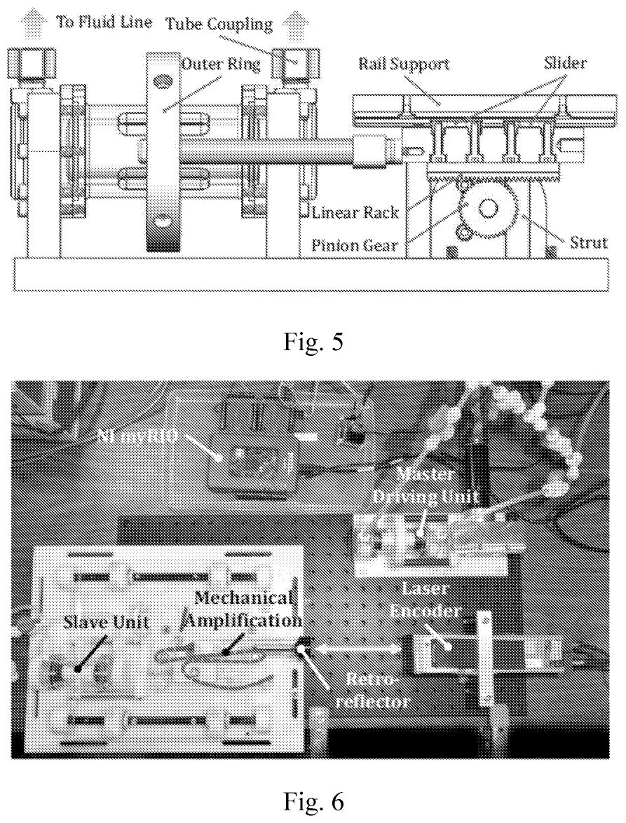

Because it is placed outside the exam room, the master unit is designed to drive the slave unit. Because the slave unit is preloaded, and because the slave unit can accommodate an additional payload, a high torque input is desired. In some implementations, the selected motor can have a gear ratio of about 86:1, which can provide sufficient torque without saturation in the motor. As indicated in FIG. 5, the torque output is transmitted through a rack and pinion mechanism to the master actuator. This mechanism is chosen for its streamline design and compactness. The moving component, where a linear rack is attached, is secured to a pair of linear guides, which can provide proper constraint of the movement with a minimum of friction. Note also that the linear rack is held by four struts so that gear teeth are in mesh regardless of torque output. The movement is then transferred to the outer ring of the master actuator by a pair of parallel rods so as to serve as an external linear bearing for the master unit.

Experimental Setup and Control Implementation:

Different respiratory profiles recorded from a 3T MRI system (MAGNETOM Prisma, Siemens, Erlangen, Germany) are deployed, and the performance is evaluated on the slave unit. It is postulated that a direct servo-loop on the master unit may achieve reduced performance because of a time delay in a fluid network, and non-smooth factors of the system, such as backlash and input deadzone. Therefore, a data-based feedforward filter and an iterative learning scheme are also introduced to more accurately replicate motion.

A respiratory motion trajectory is extracted from real-time MR images using image-based tracking. On upper abdominal MR images, an interface between the liver and the diaphragm is selected as the feature to track and the tracking search range is specified. An intensity-based multi-resolution registration scheme with a least-squares metric is applied to extract rigid-body motion of the feature throughout respiration.

Experimental Setup

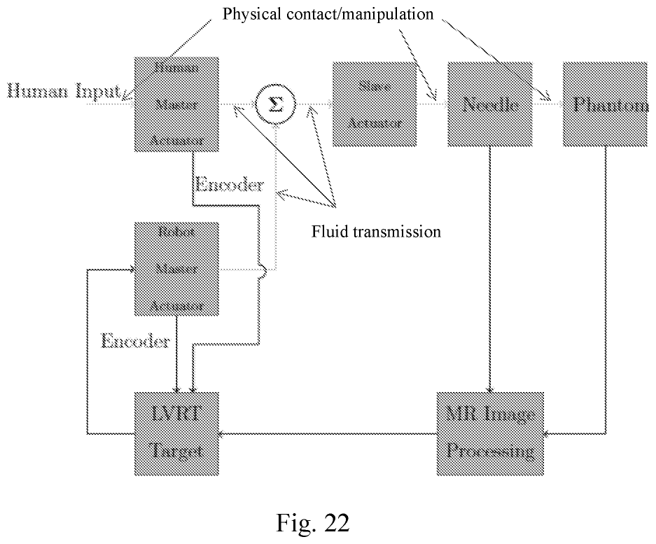

The experimental setup is depicted in FIG. 6: both the master actuator and the driving unit are mounted on an optical table. A retroreflector is attached to the slave unit and through which the displacement is measured by a laser encoder with an about 0.635 μm resolution. A real-time target (National Instrument myRIO embedded device) serves as a motion controller that is connected to and directs operation of the driving unit, and runs at an about 200 Hz loop rate. Another controller, including a processor and an associated memory storing processor-executable instructions, can be used (see FIG. 22). The laser encoder is used for training process and performance evaluation; in practical application, an offline-trained dynamic phantom can operate feedforward.

Control Implementation

Data-Based Feedforward Control Scheme:

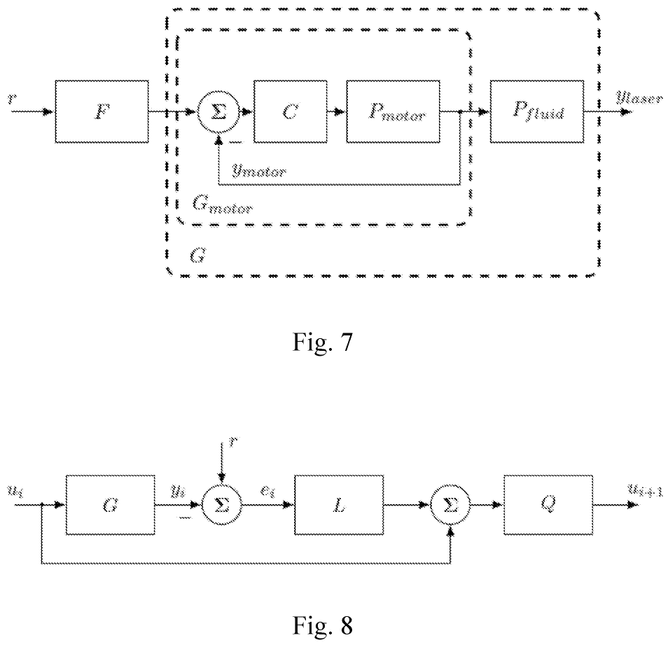

To improve the tracking performance on the slave unit, a data-based feedforward filter F is used to invert the overall dynamics G, which incorporates the dynamics of the master driving motor Gₘₒₜₒᵣ and the fluid transmission P_{fluid} as in FIG. 7. The object of this approach is to use the ILC to improve the dynamic inversion F of the system. The approach is presented as follows: First, a reference model M a zero-phase low-pass filter with d steps delay, is chosen to be an approximation of an impulse function:

G(z)F(z)≅M(z) (1)

where G(z) is the discretized overall dynamics of the pre-stabilized motor plant cascaded by the fluid transmission, and F(z) being the plant inversion.

An impulse response r(k) generated by the reference model M(z) is then used for an iterative learning process, wherein a P-type or PD-type learning filter can be used. When the process achieves convergence, the product of G(z) and converged control signal u_{∞} approximates the reference model M(z):

G(z)u_{∞}(z)≅r(z)=M(z) (2)

and this implies a stable FIR inversion can be approximated by the converged u_{∞}:

F(z)≅u_{∞}(z) (3)

With this data-driven approach, neither first-principle modelling nor system identification techniques are specified because the ILC scheme in this approach is by itself an iterative identification process of the system inversion.

Inversion-Based Iterative Learning Control.

ILC can be used to perform repeated tasks. ILC can also tolerate a certain amount of time delay and nonlinearities in the system. In this particular application, the feedforward nature of ILC lends more tractability since the system can be trained offline and no additional MR-compatible sensor is included on an end-effector.

A typical ILC block diagram is represented in FIG. 8. As can be seen, the learning error eᵢ from a previous iteration i is fed through a learning filter L, and is then used as a correction term to generate a control command for a next iteration i+1. A low-pass filter Q can be used together to suppress the high-frequency unmodeled dynamics. To summarize, the ILC learning scheme is as follows:

uᵢ₊₁(z)=Q(z)[uᵢ(z)+L(z)eᵢ(z)] (4)

The stability condition of ILC scheme can then be represented in the following inequality:

∥Q(z)[1−L(z)G(z)]∥_{∞}<1 (5)

where the left hand side of the inequality constitutes the convergence rate of the learning error.

It is noted that if the learning filter L adequately approximates the plant inversion of G, the convergence rate can be nearly zero within the bandwidth of Q, which means the error can settle to zero in one iteration. The fast convergence rate and a small learning error motivates the use of the plant inversion F as the learning filter.

The data-based inversion from the previous section can be readily used for the inversion-based ILC scheme. Since the reference model has already incorporated a low-pass filter, the update scheme (4) can be modified as the following:

uᵢ₊₁(z)=Q(z)[uᵢ(z)+z^{d}F(z)eᵢ(z)] (6)

where d steps look-ahead are used to compensate for the group delay from the reference model M

Experimental Results:

Actuator and System Verification

Benchtop system tests focused on the actuators' stiffness and repeatability. To measure stiffness, a pair of actuators were connected, and instruments affixed to their input/output shafts. A force transducer was placed between the blocked end of the output actuator and a wall, and the displacement of the input actuator is measured with a laser encoder. Stiffness is calculated from these two sets of data points using Hooke's law. To test hysteresis, the output actuator is unblocked and a second laser encoder attached. The input actuator is cycled several times, and its trajectory is examined in the phase plane. The actuator's stiffness was found to be about 10 kN/m, which should reduce losses due to system compliance, and no noticeable hysteresis was observed. To verify the design goal of strict MR-compatibility, various benchmark phantoms are scanned on a 3T MM system under the following three configurations: system removed, motion activated, and motion disabled. A table of computed SNR and image distortion measurements is shown in Table 1. No significant SNR effects or distortions introduced by either the stationary or moving system were found.

TABLE 1
MR-compatibility of System

| SNR | | | |
|---|---|---|---|
| Configuration | | Value | Change |
| System Removed | | 12.802 | — |
| Motion Disabled | | 12.719 | 0.695% |
| Motion Enabled | | 12.713 | 0.648% |
| Distortion | | | |
| Configuration | | Value [cm] | Change |
| System | Vertical | 15.127 | — |
| Removed | Horizontal | 15.170 | — |
| Motion Disabled | Vertical | 15.144 | 0.112% |
| | Horizontal | 15.132 | 0.251% |
| Motion Enabled | Vertical | 15.042 | 0.562% |
| | Horizontal | 15.183 | 0.086% |

Tracking Results

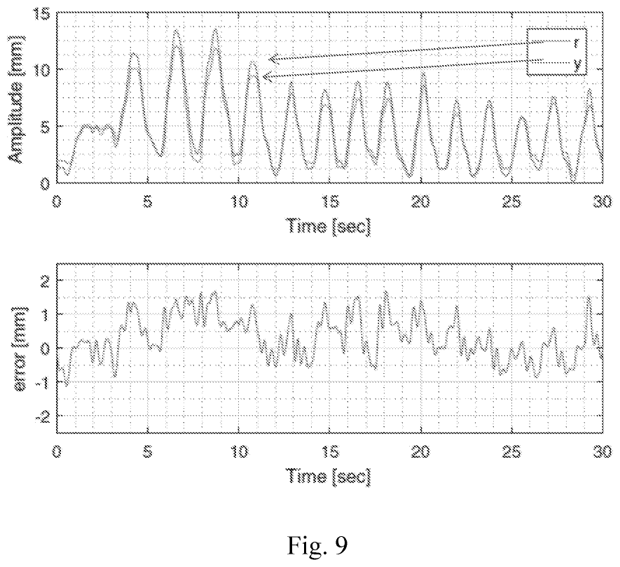

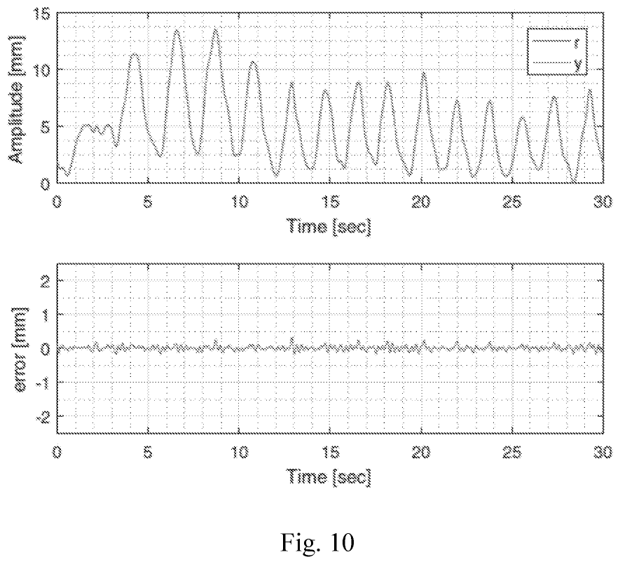

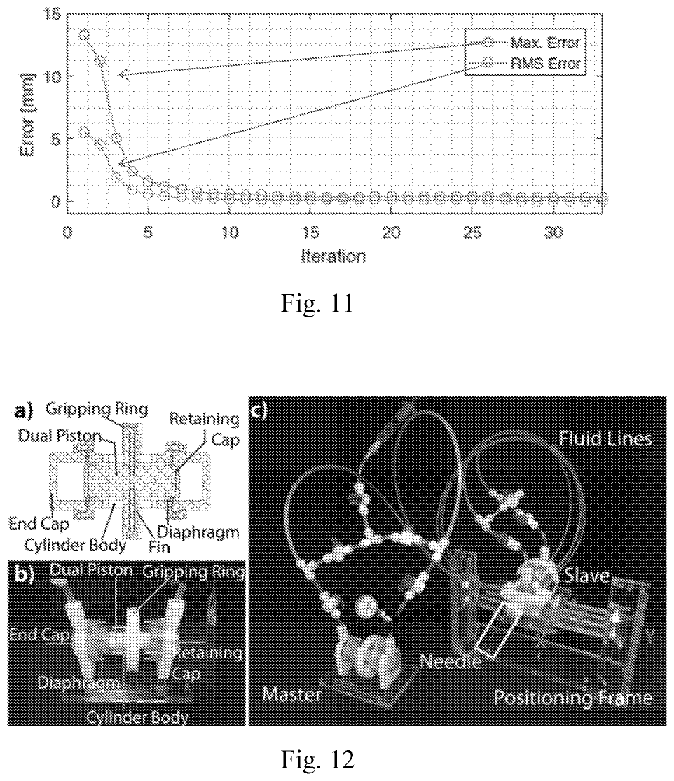

With the system's MR-compatibility verified, further testing is conducted on the benchtop. First, the naive master-side PI-only scheme is tested on a reference profile. The tracking performance is poor, especially at direction changes, and there is a large uncompensated group delay. It is noted that the hydraulic system has dynamics that should be accounted. Next, the feedforward dynamic inversion is applied (FIG. 9). The group delay is compensated, but the tracking at direction changes is not significantly improved over PI. ILC is then applied with the same dynamic inversion as the learning filter, which resulted in much improved tracking performance (FIG. 10). The convergence plot for ILC is shown in FIG. 11. Results from these experiments are summarized in Table 2. System repeatability was verified by running the learned feedforward reference to the master's inner PI loop various times over the course of a 24-hour period. For all tests attempted, the tracking performance was similar to the original result shown in FIG. 10.

With the promising results from the unamplified system, experiments were performed with the mechanically-amplified motion platform. A different, larger-amplitude profile is selected for training, and the results are shown in Table 2. Given its superiority, the inversion-based ILC control scheme was implemented on the amplified system. The results achieve sub-millimeter tracking (about 0.198 mm RMS, about 0.664 mm MAX), which is adequate for various applications. An examination of the spectrum of the error signal indicates that an inversion with higher bandwidth may be able to achieve performance comparable to the unamplified system.

TABLE 2
Summary of Tracking Control Performance. FF-ILC1 uses ILC for
the control for tracking an impulse basis function and constructs the
feedforward control for the reference trajectory by linear combination,
and FF-ILC2 uses ILC to directly track the reference trajectory by
applying the learning filter obtained from the FF-ILC1.

| Controller | RMS Error [mm] | Max. Error [mm] |
|---|---|---|
| PI | 0.973 | 2.430 |
| PI + FF-ILC1 | 0.652 | 1.687 |
| PI + FF-ILC2 | 0.072 | 0.328 |

Conclusions:

Some embodiments of a strictly MR-compatible system capable of tracking pre-recorded respiratory motion trajectories are presented herein. The results include a fully MR-compatible, repeatable, mechatronic system capable of about 25 mm travel in one degree of freedom. Experimental results are presented from the implementation of three control schemes: master-side PI, feedforward dynamic inversion, and inversion-based Iterative Learning Control—the latter two demonstrating feasibility of feedforward schemes in this application. The results further include tracking results from additional profiles with varying bandwidth to demonstrate system flexibility. Tracking results are presented from a system implementation with mechanical stroke amplification, capable of about 50 mm travel. Further improvements can include a refinement of the mechanically-amplified implementation and the exploration of in-bore applications for this device (e.g., manual or automated tracking and targeting of simulated lesions).

MRI-Guided Targeted Needle Placement During Motion Using Hydrostatic Actuators

Overview:

MR imaging (MM) has advantages for guiding minimally invasive interventions. However, a constraint is that the narrow gantry space of MM scanners restricts access to a patient by a physician. In this disclosure, a master-slave rolling-diaphragm hydrostatic actuator system is evaluated for MM-guided minimally invasive interventions in targets experiencing motion. The effects of the system on imaging as well as its input-output response are characterized. The accuracy and time efficiency of human-operated remote-controlled targeted needle placement using the actuator system is evaluated with respect to a comparative approach in both static and dynamic targets under MRI guidance.

Methods.

The effects of the actuator system on MRI SNR and geometric distortion are evaluated in phantoms. The ability of the actuator system to transmit displacement input from a master outside the scanner to output at a slave inside the scanner was characterized. An MM-conditional motion phantom is formed to provide 10-mm diameter targets without motion (static) and with reproducible motion (dynamic). Using both a Step-and-Shoot (SS) method and an Actuator-Assisted (AA) method, an operator performed MM-guided targeted needle placement in the static (n=12) and dynamic (n=12) targets. The needle-to-target error (NTE) and time for each procedural step (planning, entry point, insertion, confirmation) are recorded. Non-parametric tests are used to compare differences in the means (Mann-Whitney U Test for static and Wilcoxon Sign Ranked Test for dynamic) and group variance (Brown-Forsythe Test) between the SS and AA methods for procedural times as well as NTE. Statistical significance is considered at the p<0.05 level.

Results.

The hydrostatic actuator system exhibited negligible impact on MRI SNR and geometric fidelity. The system provided a linear master-slave input-output response in both the push and pull directions. For the static targets, both the SS and the AA methods were able to achieve similar NTE (NTE_{SS}=1.33±0.66 mm; NTE_{AA}=1.27±0.50 mm). Once the entry point was verified, AA involved about half the insertion time (IT) to reach the target than SS (IT_{SS}=5.16±5.86 min, IT_{AA}=2.42±3.02 min, p=0.024). In dynamic targets, the AA method was more accurate (p=0.015) and precise (p=0.016) with NTE_{SS}=3.29±1.823 mm and NTE_{AA}=1.82±1.04 mm. The total procedure time (TT) using AA was reduced by 25% compared to SS (TT_{SS}=36.34±9.46 min, TTAA=25.99±5.28 min, p=0.005).

Conclusion.

A hydrostatic actuator driven motion control system is presented to support MRI image tracking by providing specified motion as ground truth for MR image calibration, registration, and tracking. It is also useful for MM-guided minimally invasive interventions during motion. The actuator system had negligible effects on MM SNR and distortion, and achieved linear input-output response. Using the actuator system, a human operator was able to achieve targeted needle placement with significantly improved accuracy (mean NTE<about 2 mm) and reduced procedure time (about 25% less total time) compared to a SS method in targets with motion. The rolling diaphragm hydrostatic actuator system can allow physicians to remotely perform real-time MM-guided interventions even while the targets are in motion.

Introduction:

Minimally invasive interventions, including targeted biopsy and focal ablation, are a safe and effective way to diagnose and treat localized cancers in the liver, kidney, and other vital abdominal organs. Due to steady advancements, these treatments are now used as first line therapy in selected patients and cancer types. The least invasive form of interventions is the class of percutaneous interventions where needles and catheters are used without an incision, resulting in decreased complication rates, reduced recovery times, and higher patient eligibility. The foundation of these procedures is targeted placement of a device (e.g., needle). Targeting accuracy within millimeters is desired, especially if the target is small, or close to another organ, vessel, or nerve.

Since direct optical visualization for procedural guidance is typically not possible, percutaneous interventions depend on other imaging methods for guidance. Currently, the vast majority of interventions are guided by ultrasound and CT. Although ultrasound and CT are both practical and widely available, there are constraints to their capabilities which impedes the overall effectiveness and widespread adoption, restricting their availability for many patients. Ultrasound can fail due to inadequate acoustic windows, and poor visualization of deeply located tissues. Gas bubbles generated by ablation can further decrease ultrasound visibility of lesions. For CT, intravenous contrast is specified to visualize many abdominal tumors, but the contrast can be administered once and tumor visualization may be transient, which is inadequate for an entire intervention. Real-time imaging throughout a procedure is also typically not feasible with CT due to radiation concerns.

MRI has advantages for guiding interventions, especially in areas with motion, such as the abdomen. It is the desired modality for detecting tumors in organs such as the liver and kidney, and may be the sole modality where a tumor is visible. MRI does not involve ionizing radiation and therefore can be used continuously throughout an entire procedure, offering the ability of real-time image guidance. Real-time guidance is a major advantage in the abdomen due to inherent motion of intra-abdominal organs. There are, however, several challenges to be overcome; primarily, a narrow gantry space of most MRI scanners restricts access by a physician to a patient. MRI scanners typically have a longitudinal distance of about 60-100 cm from an entrance to a center of a scanner bore and a cross-sectional bore diameter of about 50-70 cm.

Remotely-controlled actuator systems are a promising strategy to overcome the challenge of physical space restrictions in the MM scanner. Due to the strong electromagnetic fields in the MM environment, electromagnetic actuation mechanisms and conductive/magnetic materials are not acceptable. Hydrostatic actuation is a desirable approach. Hydrostatic actuators have low backlash, are back-drivable, have force feedback, and do not specify shielding of electronics and lines to prevent MR image distortion. Therefore, hydrostatic actuation is advantageous for MRI-guided interventions.

Once a remotely-controlled actuator system is implemented, targeted needle placement is a step in evaluating its effectiveness. The actuator system should be able to precisely and accurately guide the needle to a predetermined target, including under motion or dynamic conditions. When a target (e.g., tumor) is affected by motion (e.g., respiratory motion) it increases the difficulty of device placement, since the target's position is constantly changing. Therefore, in order to fully investigate the effectiveness of an actuator system for needle placement in regions where targets experience motion (e.g., the abdomen), it should be examined under dynamic conditions.

A remote-controlled hydrostatic actuator system is developed for MRI-guided minimally invasive interventions. In this disclosure, the hydrostatic actuator system is evaluated for MRI-guided minimally invasive interventions in targets experiencing motion. Investigation is made on the effect of the actuator system on imaging quality as well as its master-slave input-output response. Using a specially designed dynamic motion phantom, evaluation is made of the accuracy and time efficiency of human-operated remote-controlled targeted needle placement using the actuator system under MRI guidance. The performance of our actuator system is compared to a comparative approach (no actuator) in both static and dynamic targets.

Materials and Methods:

Hydrostatic Actuator System Design

In order for actuators to be used in MRI-guided interventions, the actuators should not notably affect the quality of an image, and the MR environment should not affect the operation of the actuators. Therefore, ferromagnetic materials and electromagnetic components were excluded from the design. In addition, hydrostatic actuators should balance competing specifications of avoiding fluid leakage and low friction, which is a challenge for piston-cylinder actuators. To meet the aforementioned design constraints, a master-slave hydrostatic actuation system is developed using a pair of low-pressure water-based rolling diaphragm hydrostatic actuators (FIG. 12a). Rolling diaphragm actuators can have slightly larger friction than certain piston-based actuators, but can be fully sealed to avoid fluid leakage. The actuators were constructed of polyoxymethylene (piston, fins, compression fittings and gripping ring) and acrylic (cylinder body and end caps) (FIG. 12b). The diaphragms were cast in custom molds using silicone and reinforced with fabric mesh. The stroke of the hydrostatic actuators in the system was about 25 mm. The slave actuator was designed to fit in a 60-cm diameter MRI scanner bore, and was outfitted with a needle holder to insert an MR-conditional needle during a procedure. The slave actuator was positioned and stabilized using an acrylic frame, which is adjustable in both the horizontal (X) and vertical (Y) directions (FIG. 12c).

In order to transmit displacement across the actuator pair, the operator moves the gripping ring on the master actuator. When the gripping ring is advanced, the water is transferred through fluid lines, transmitting an input force and displacement to the gripping ring of the slave actuator. The gripping ring of the slave actuator is attached to the needle holder, advancing the needle. For this investigation, the actuator system was constructed to be manipulated in one degree-of-freedom (DoF) for needle insertion along Z. The horizontal (X) and vertical (Y) positions were manually adjusted using the frame. The actuator pair was designed to achieve a linear master-slave response with input-output ratio of about one.

MRI-Guided Targeted Needle Placement

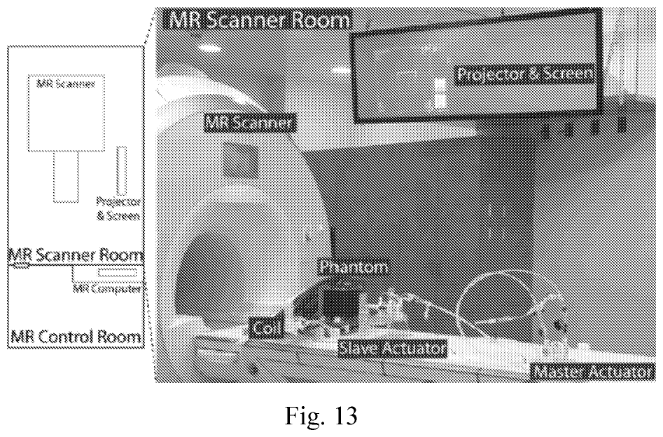

MRI-guided targeted needle placement is performed in a specially designed interventional MM suite with a 3T whole-body MRI system (MAGNETOM Prisma, Siemens, Erlangen, Germany) (FIG. 13). All experiments were performed in this suite using a 32-channel body array coil. In order for the operator to visualize and perform an interventional procedure, intra-procedural MR imaging feedback was used to depict the position of the target as well as the needle during the entire procedure. To provide real-time visualization, the MM scanner room was outfitted with shielded projectors and a screen to display MR images.

The MM-guided targeted needle placement experiments were modeled closely after clinical image-guided procedures by two abdominal interventional radiologists (over 20 years of experience in image-guided interventions). An operator was trained by the two interventional radiologists to consistently perform all experiments. For Actuator-Assisted (AA) interventions, the slave actuator was placed next to the object inside the scanner while the master actuator was placed at the end of the patient table to allow remote control by the operator inside the scanner room. Details regarding the Step-and-Shoot (SS) method and the AA method, for both static and dynamic targets, are presented later in this section. In order to familiarize the operator and research team to both targeting methods as well as the MR imaging sequences used in the experiments, there was a series of initial training experiments led by the interventional radiologists. The training experiments used n=5 targets for both the SS method and the AA approach. Following every training target, the operator visualized the final position of the needle tip in relation to the target on high-resolution MR images, and compared it to what was observed in the real-time MR images, and used the information to improve accuracy of needle placement at the next target. The operator was trained for both static and dynamic targets.

Static and Dynamic Phantom Design

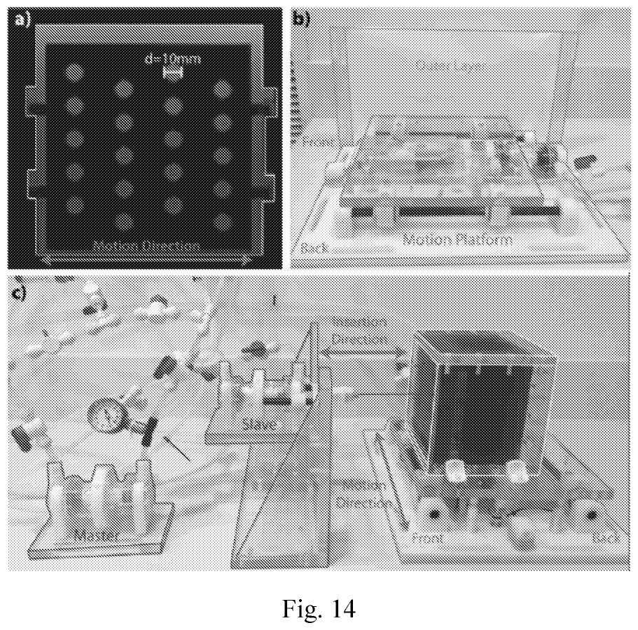

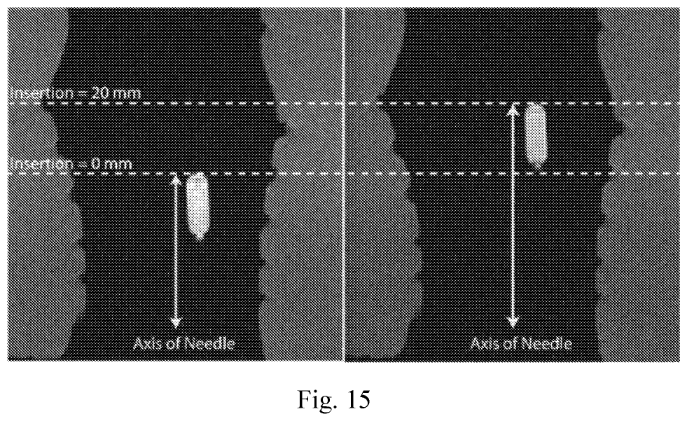

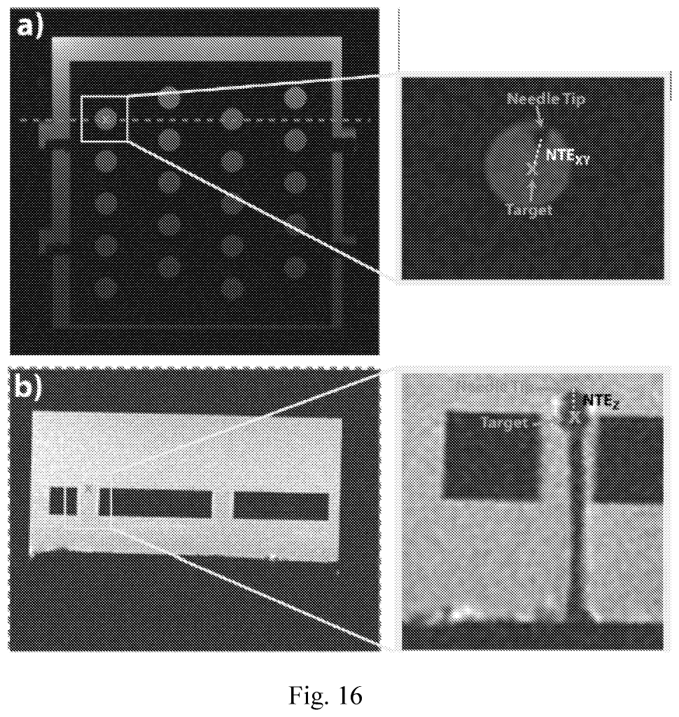

An acrylic phantom with a targeting plate was constructed (FIG. 14a). The targeting plate was formed by drilling twenty about 10-mm diameter holes in an about 12.5-mm thick sheet of acrylic. The about 10-mm diameter was chosen as it represents a clinically relevant target size for percutaneous interventions. The targeting plate was then inserted into the phantom and the entire targeting phantom was filled with a dark gelatin solution (to obscure direct visualization) and cooled.

The phantom was secured to a custom-designed motion platform to allow the evaluation of targeted needle placement in dynamic targets. The motion platform was designed and constructed using the same type of rolling diaphragm hydrostatic actuators as in the actuator system (FIG. 12a). Using a computer-controlled motor, the motion platform was programmed to reproduce an oscillatory (e.g., sinusoidal) waveform of about 0.3 Hz and peak-to-peak displacement range of about 20 mm (similar to a human respiratory cycle). The motion was set to be substantially perpendicular to the axis of the needle (FIG. 14c). In order to emulate the conditions for organ movement in the body, an outer layer with a sheet of paper was secured in front of the motion phantom to represent skin and abdominal muscle, which does not follow the breathing motion of the organs and holds the needle stationary (FIG. 14b).

The motion platform was equipped with a “breath hold” feature, which simulated a traditional breath hold scheme applied in interventional procedures. The operator prompted the phantom to perform a “breath hold” in a position close to end expiration before imaging and needle insertion. The breath hold position is manually selected, so there are natural variations in the breath hold position, as seen in clinical procedures. The breath holds were at most about 20 seconds in order to mimic realistic patient abilities.

System Characterization:

Imaging Assessment

In order for the actuator system to be used in the MR environment, it is designed to have minimal impact on the MR image quality, including imaging artifacts, reduction in the SNR, or distortion of an image. To evaluate the impact on image quality, two phantom experiments were performed. First, a spherical phantom was set up in the MRI scanner bore under a body array coil with the slave actuator positioned beside it. The set up was imaged using a high-resolution 3D Gradient Echo (GRE) sequence and 2D multi-slice Turbo Spin Echo (TSE) sequence (parameters in Table 3), with the actuator and phantom in the imaging plane. Each sequence was acquired twice and the difference method was used to calculate SNR. The actuator was then removed with care to preserve the physical setup (e.g., coil position) and the scans were repeated. The SNR was calculated for each sequence, both with and without the actuator, on three distinct slices and the average percent difference was calculated. The images were also visually examined for artifacts.

Next, the American College of Radiology (ACR) MRI quality control phantom was used in place of the spherical phantom, and the actuator was placed next to the grid of the ACR phantom. The set up was again imaged using both the 3D GRE and 2D TSE sequences, with the actuator and phantom in the imaging plane. The actuator was then carefully removed and the scans were repeated. The length of the central horizontal and vertical grid lines, as well as the angle between the central grid lines were measured for each sequence, both with and without the actuator, and the percent difference was measured to assess distortion. The images were also visually examined to assess distortion.

TABLE 3
Sequence parameters for experiments.

| | System Characterization | | Targeted Needle Placement | |
|---|---|---|---|---|
| | 3D GRE | 2D TSE | 3D GRE | 2D GRE |
| TE/TR (ms) | 4.2/9.6 | 15/2290 | 4.08/9.2 | 3.17/5.88 |
| FOV (mm²) | 280 × 280 | 280 × 280 | 300 × 300 | 300 × 300 |
| Resolution (mm³) | 0.5 × 0.5 × 1 | 0.5 × 0.5 × 1 | 0.5 × 0.5 × 1.5 | 2.4 × 2.4 × 5 |
| Flip Angle (°) | 25 | 180 | 10 | 12 |
| Slices | 44 | 20 | 104 | 1 (single-slice) |
| | | | | 2: sagittal and coronal (multi-slice) |
| Scan Time (min:sec) | 2:10 | 1:08 | 2:08 | 1.3 sec/image (single-slice) |

Master-Slave Input-Output Response

In order to assess the linearity of the actuator system for transmitting displacement from master input to slave output, the actuator pair was initially verified on bench top. The linearity of the actuator system was then evaluated in the MRI scanner bore. In this case, the master actuator was placed in the control room and connected to an electronic computer-controlled motor to ensure precise input position increments. The fluid lines and slave actuator were passed through a waveguide into the scanner room and placed on the patient bed. Contrast beads (MR-Spot, Beekley, 1.5 cm) were placed along the edge of an MR-conditional aspiration needle (Invivo, 16G, 10 cm) to visualize needle position, and the needle tip was positioned between two water bottles to have adequate signal for imaging. The needle was inserted along the stroke of the actuator system, advancing by about 2-mm input increments, and the displacement was measured under MM using high-resolution 3D GRE and 2D multi-slice TSE (Table 3) sequences after each increment. This procedure was repeated as the master actuator was moved in the opposite direction (e.g., needle was retracted). The position of the contrast bead at its initial position was taken as the baseline (FIG. 15), and the position of the needle and contrast bead following every insertion or retraction was measured and subtracted from the baseline. The motor-controlled input insertion/retraction position and the corresponding output needle position was then plotted. The linear regression for each of the datasets was calculated.

Targeted Needle Placement in Static Targets Under MM Guidance

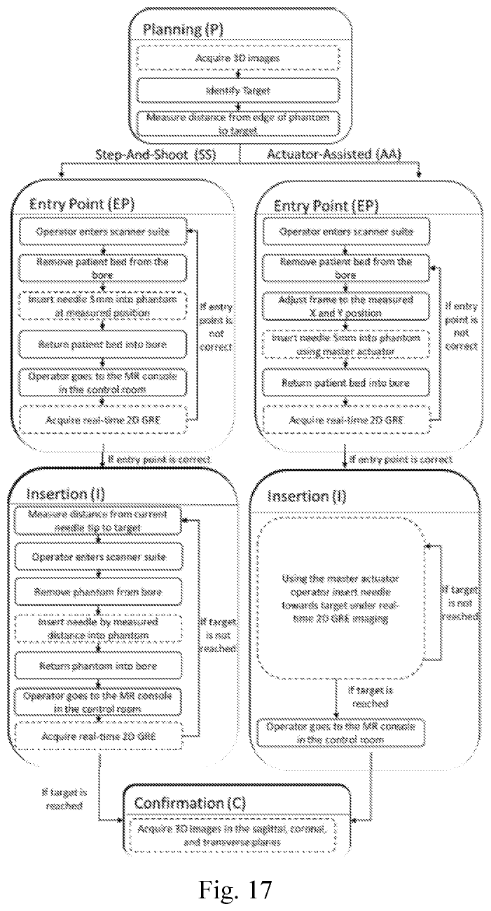

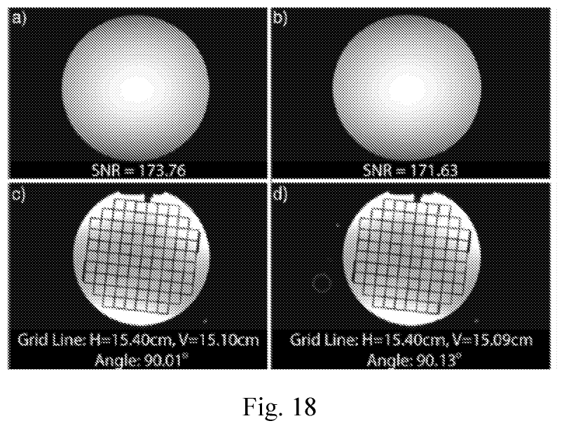

The targeting phantom was secured onto the motion platform, set to the static motion state (no motion), and placed on the MRI patient bed. The operator was instructed to maneuver an MR-conditional aspiration needle (Invivo, 18G, 10 cm) to a target, defined as the center in the X and Y directions, and far edge in the Z direction (FIG. 16), of a selected 10-mm diameter hole, using either the SS or AA approach. Both techniques have a total of n=5 training targets followed by n=12 distinct targets, which were matched according to relative position and difficulty. Both techniques were separated into four main steps: Planning, Entry Point, Insertion and Confirmation (FIG. 17).

For both SS and AA, the Planning and Confirmation steps are identical. The Planning step began with a 3D GRE planning scan (Table 3). From the planning scan, the target was identified and the operator measured the distance from the edge of the phantom to the target on the MR images at the console computer. The Confirmation step included a set of three high resolution 3D GRE scans (same protocol as the planning scan) in the sagittal, coronal, and transverse planes. Entry Point and Insertion steps, however, differ in the two targeting schemes, as explained next.

Step-And-Shoot: For the Entry Point step, the operator entered the scanner room, removed the patient bed from the bore, and used the measurements from the Planning step to slightly insert the needle by hand into the phantom at the expected entry point. The bed was returned into the bore as the operator returned to the control room and the phantom was imaged using a “real-time” multi-slice (sagittal and coronal planes) 2D GRE sequence (Table 3). If the operator decided that the entry point should be corrected, the prior steps were repeated until the entry point was in the correct position. Once the entry point was in the correct position, the operator moved to the Insertion step.

For Insertion, the operator measured the distance to the target on the latest image set acquired and returned to the scanner room as the patient bed was removed from the bore. The operator inserted the needle toward the target. The bed was returned into the bore, the operator returned to the control room, and the phantom was imaged using the same real-time sequence. If the operator decided that the insertion distance should be corrected, the offset was measured and the operator returned to the scanner room and adjusted the insertion. This was repeated until the operator believed that the target was reached and the operator moved to the Confirmation step.

Actuator-Assisted:

For the Entry Point, the operator entered the scanner room, removed the bed from the bore, used the measurements from the planning step to adjust the frame to the correct position. When the frame was in the correct position, the operator slightly inserted the needle into the phantom at the expected entry point using the master actuator. The bed was returned into the bore and the phantom was imaged using the same real-time multi-slice (sagittal and coronal planes) 2D GRE sequence while the operator remained in the scanner room viewing the images in real-time. If the operator decided the entry point should be corrected, the phantom was removed from the bore and the frame adjusted. This was repeated until the entry point was in the correct position. Once the entry point was in the correct position, the operator moved to the Insertion step. For Insertion, the operator stayed by the patient bed and used the master actuator to insert the needle to the target with visual feedback from the real-time 2D GRE sequence, stopping once the target was reached and left the scanner room.

Targeted Needle Placement in Dynamic Targets Under MRI Guidance

The targeting phantom and outer layer were secured onto the motion phantom and the motion phantom was programmed with the prescribed sinusoidal motion waveform. The operator was again instructed to maneuver the MR-conditional aspiration needle to a target using either the SS or AA approach. Using each targeting method, the operator first targeted n=5 training targets, followed by n=12 targets. In order to improve the comparison, both techniques targeted the same n=12 targets over multiple days, during which the gelatin in the phantom was replenished to eliminate track marks from previous attempts. Both techniques were again separated into the same four main steps (FIG. 17).

For both targeting techniques, the motion platform was placed on an indefinite breath hold at end expiration over the entire imaging duration for the Planning and Confirmation steps. All breath holds were depicted as dotted lines in FIG. 17. Following the 3D GRE planning scan, the motion began as the target was identified. The operator measured the distance from the contrast beads placed on the outer layer to the target on the console. For the Confirmation step, the same set of three static 3D GRE scans was acquired under an indefinite breath hold.

For the dynamic Entry Point and Insertion steps, the operator requested a breath hold (about 20 sec) during imaging and needle insertion, while following the same steps as the static case for both targeting methods. The imaging for the dynamic case was done with real-time single-slice 2D GRE sequences (Table 3), with multiple measurements (n=110) to ensure adequate imaging time for the procedure. The imaging was first acquired in the sagittal plane along the target to view the Y offset of the entry point, followed by the coronal plane along the target for the X direction. For the SS method, the operator asked for a breath hold (about 20 sec) from the control room before any imaging began, and from the scanner room before inserting the needle. For the AA method, the operator viewed the real-time images in the scanner room and used the images to decide when to request a breath hold (about 20 sec) for positioning and needle insertion.

Statistical Analysis of MRI-Guided Targeted Needle Placement

Targeted needle placement performance was evaluated in terms of time efficiency, accuracy and precision (group variance). Since the training targets were used to accustom the operator to both the targeting techniques as well as the imaging sequences used in the experiment, the data from those targets were not used in the data analysis.

For each experiment, the time of each step and total time (from the first planning scan to the final confirmation scan) were recorded. The needle targeting accuracy and precision were characterized in terms of needle-to-target error (NTE). The NTE 15 specified as the X-Y-Z Euclidean distance in mm between the final needle tip position and the center of the target as (see FIG. 16):

(NTE=\sqrt{NTE_{XY}²+NTE_{z}²})

Since the data was non-normal, non-parametric tests were used to compare differences in the means and group variance between the SS and AA data for each timing step as well as all of the NTEs. SPSS (IBM Corporation, Armonk, N.Y.) software was used for the statistical analyses. For the static targets, the Mann-Whitney U Test was applied since SS and AA methods used different targets. For the dynamic case, since the same targets were used, the Wilcoxon Sign Ranked Test was used. The Brown-Forsythe Test was used to evaluate differences in the group variance. Statistical significance was considered at the p<0.05 level for all tests.

Results:

System Characterization

Imaging Assessment

No visible image artifacts were observed with the actuator inside the MRI scanner bore (FIG. 18). The SNR difference for the sphere phantom with and without the actuator is on average about 2.2% (difference in GRE SNR=about 1.2%, difference in TSE SNR=about 3.15%). The ACR phantom images showed no distortion. The grid line length difference with and without the actuator was on average about 0.09% (about 0.04% on GRE, about 0.14% on TSE). The grid angle difference with and without the actuator was on average about 0.11% (about 0.06% on GRE, about 0.16% on TSE).

Master-Slave Input-Output Response

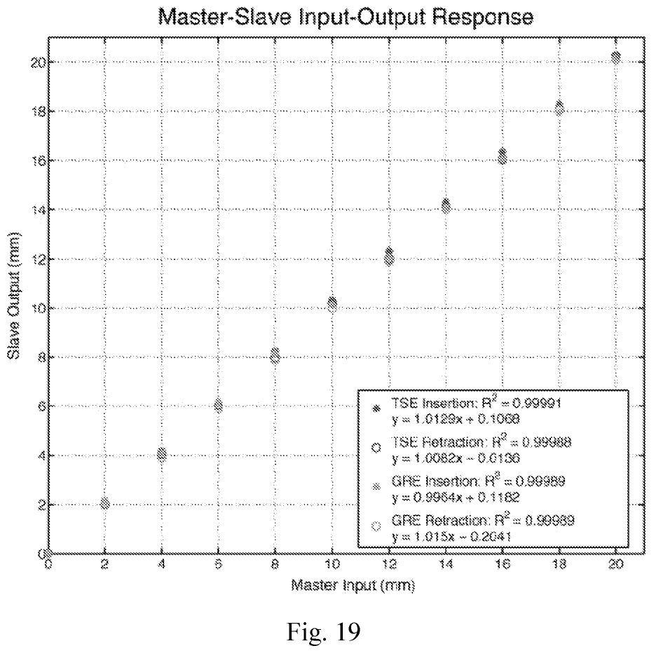

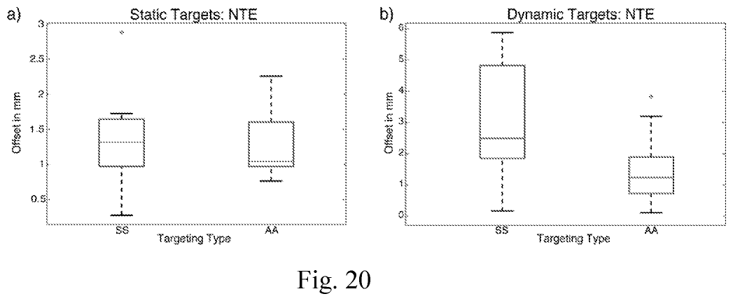

The input of the master versus output of the slave displacement can be seen in FIG. 19. From measurements on 3D GRE, the slave actuator moved about 1.01 mm to every about 1 mm displacement applied by the master, and on 2D TSE this ratio was about 1.00 mm to every about 1 mm displacement. The linear correlation is nearly identical for both the push (insert) and pull (retract) directions.

Targeted Needle Placement in Static Targets Under MM Guidance

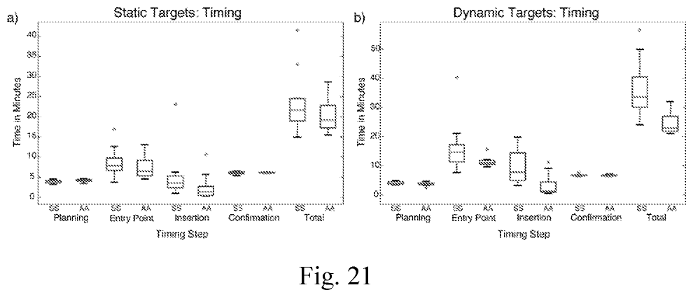

The mean, standard deviation (STD), and statistical comparison results for the static experiments can be found in Table 4. Both the SS and the AA methods were able to achieve similar NTE (NTE_{SS}=1.33±0.66 mm; NTE_{AA}=1.27±0.50 mm) (FIG. 20a). For total targeting time (TT), the AA method was on average about 15% faster than SS (TT_{SS}=23.58±7.22 min, TT_{AA}=20.23±3.86 min) (FIG. 21a, Table 4). This difference in total targeting time mainly came from the Insertion time (IT), since the planning scan, entry point determination, and confirmation scan were almost identical between the two targeting schemes. On average the AA Insertion time was about 2 times faster than the SS method (IT_{SS}=5.16±5.86 min, IT_{AA}=2.42±3.02 min). The Insertion time was the sole step with a significant difference (Mann-Whitney U test p=0.024).

Targeted Needle Placement in Dynamic Targets Under MRI Guidance

When targets experienced motion, the NTE of the SS and AA methods increased (NTE_{SS}=3.29±1.823 mm; NTE_{AA}=1.82±1.04 mm) (FIG. 20b, Table 5). As seen in the plots, the NTE for AA was significantly lower with a reduced variance compared to SS (p=0.015 and p=0.019 respectively). For the total targeting time, AA was on average about 25% faster than the SS method (TT_{SS}=36.34±9.46 min, TT_{AA}=25.99±5.28 min) (FIG. 21b, Table 5). In this case, the difference in targeting time came from the both the Entry-Point time (EPT) and Insertion time (IT). On average the EPT was reduced by about 30% using AA (EPT_{SS}=16.07±8.57 min, EPT_{AA}=11.04±1.75 min), and the IT using AA was about 2 times faster than the SS method (IT_{SS}=9.60±5.85 min, IT_{AA}=4.59±5.71 min). The EPT, IT and TT also had significantly different mean values (p=0.028, 0.010, and 0.005 respectively) and the IT and TT had significant differences in group variance (p=0.045 and 0.004 respectively).

TABLE 4
The mean, standard deviation (STD), and statistical test results of NTE and
timing for SS and AA for static targets.

| | | | Planning | | Entry-Point | | Insertion | | Confirmation | | Total Time | |
|---|---|---|---|---|---|---|---|---|---|---|---|---|
| | NTE (mm) | | Time (min) | | Time (min) | | Time (min) | | Time (min) | | (min) | |
| Static | SS | AA | SS | AA | SS | AA | SS | AA | SS | AA | SS | AA |
| Mean | 1.33 | 1.27 | 3.90 | 4.17 | 8.45 | 7.50 | 5.16 | 2.42 | 6.07 | 6.15 | 23.58 | 20.23 |
| STD | 0.66 | 0.50 | 0.43 | 0.34 | 3.63 | 2.77 | 5.86 | 3.02 | 0.34 | 0.14 | 7.22 | 3.86 |

| | | | | | | |
|---|---|---|---|---|---|---|
| Mann-Whitney U | 0.664 | 0.128 | 0.443 | 0.024* | 0.713 | 0.198 |
| p value | | | | | | |
| Brown-Forsythe | 0.815 | 0.107 | 0.481 | 0.169 | 0.513 | 0.175 |
| p value | | | | | | |

Statistically significant results are indicated with an asterisk.

TABLE 5
The mean, standard deviation (STD), and statistical test results of NTE and
timing for SS and AA for dynamic targets.

| | | | Planning | | Entry-Point | | Insertion | | Confirmation | | Total Time | |
|---|---|---|---|---|---|---|---|---|---|---|---|---|
| | NTE (mm) | | Time (min) | | Time (min) | | Time (min) | | Time (min) | | (min) | |
| Dynamic | SS | AA | SS | AA | SS | AA | SS | AA | SS | AA | SS | AA |
| Mean | 3.29 | 1.82 | 4.00 | 3.72 | 16.07 | 11.04 | 9.60 | 4.59 | 6.68 | 6.65 | 36.34 | 25.99 |
| STD | 1.68 | 1.04 | 0.53 | 0.60 | 8.57 | 1.75 | 5.85 | 5.71 | 0.31 | 0.28 | 9.46 | 5.28 |

| | | | | | | |
|---|---|---|---|---|---|---|
| Wilcoxon | 0.015* | 0.136 | 0.028* | 0.010* | 0.433 | 0.005* |
| Signed Rank | | | | | | |
| p value | | | | | | |
| Brown-Forsythe | 0.019* | 0.228 | 0.070 | 0.045* | 0.795 | 0.004* |
| p value | | | | | | |

Statistically significant results are indicated with an asterisk.

Discussion:

The hydrostatic actuator system exhibited negligible impact on MR image SNR and geometric fidelity, allowing for utilization in the MM scanner bore to allow MRI-guided interventions. The master-slave actuator pair provided a linear input-to-output displacement response in both the push and pull directions. This is desired for the operator to be able to accurately maneuver the needle to target locations and allows more intuitive transition to a human-operated remote-controlled approach. The linearity of the actuator pairs is also desired for implementations of computer-assisted interventions.

An advantage of the evaluation is that the MRI-guided targeted needle placement experiments were closely modeled after actual clinical procedures. In particular, the evaluation explicitly considered the effects of motion in a controlled setting. To systematically evaluate the actuator system for both static and dynamic conditions, a dynamic targeting phantom is devised, and experiments are performed under reproducible motion conditions. The system was used to successfully guide a needle to all targets under real-time MM in both the static and dynamic cases, for both the SS and AA methods.

The position of the target in the MM scanner bore was beyond reach, therefore the operator could not perform free-hand needle insertion under real-time MRI guidance. Although there are shorter bore MM scanners, those systems are not as widely available and have limited field-of-view. Even with a shorter bore, targets deep in the body or reachable by oblique trajectories may still be hard to reach. The results demonstrated that the actuator system can potentially extend the operator's reach for MRI-guided interventions at the center of the MRI scanner.

Clinically, physicians can target lesions as small as about 5-10 mm, which specifies achieving NTE<about 2.5 mm. The results demonstrate the feasibility for the actuator system to attain this accuracy in both static and dynamic cases. For static targets, both the SS and AA methods achieved mean NTE<about 2.5 mm. In one case did the NTE_{SS} exceed about 2.5 mm. Although both targeting methods were able to achieve the clinically relevant NTE performance in the static case, targets in the clinical setting, especially for abdominal organs, are not static.

When targets experienced motion in the dynamic experiments, NTE<about 2.5 mm was more difficult to attain. The mean NTE_{SS} was about 3.29 mm, exceeding the clinically relevant threshold. AA targeting was able to attain a significantly lower mean NTE (about 1.82 mm, p=0.015), with just two targets exceeding the about 2.5 mm threshold. The AA targeting method also had a significantly lower variation (p=0.019), indicating improved reproducibility of NTE across trials. The increased accuracy in the NTE demonstrates that continuous real-time visualization while inserting the needle is valuable in a dynamic setting, where motion can affect the needle and target position.

In a clinical setting, longer procedure times increase complication risk for patients and reduce adoption of MM-guided interventions. Therefore, reducing procedure time for MM-guided interventions is advantageous for its clinical translation. In the static targets, the Insertion Time was significantly lower for the AA method compared to SS, (mean IT_{SS}=about 5.16 min, mean IT_{AA}=about 2.42 min, p=0.024). This indicates that once the entry point was verified, AA involved about half the time to reach the target compared to SS in a static target. For SS, the operator inserted the needle step-wise, leaving the scanner room for imaging after every insertion; while for AA it was a single step, leading to a decrease in the insertion time.

Under dynamic conditions, the significant time reduction extends to the Entry Point and Total Time, in addition to Insertion Time, for AA targeting. When the targets were in motion, the operator was able to visualize the target's position during the entire motion cycle, and use that knowledge to anticipate when the target is aligned with the needle trajectory, leading to a reduction in the procedure time. This knowledge results in both a significant reduction in total time (mean TT_{SS}=about 36.34 min, mean TT_{AA}=about 25.99 min, p=0.005) and less variation (p=0.004).

With advances in MM, tumors can be detected at an earlier stage and at smaller sizes. In order to target these tumors during minimally invasive interventions, higher accuracy is desired. The actuator system demonstrates the ability to achieve low NTE even during motion and has potential to allow earlier diagnosis and better treatment for early-stage tumors. As smaller tumors are being targeted, there may be an increase in targeting difficulty, leading to an increase in procedure time. Also, in an interventional procedure there are typically multiple regions of a tumor to be biopsied or ablated, increasing the number of targets. The procedure may span for multiple hours with multiple insertion steps. Therefore, the potential of the system to reduce the time of each insertion step by about 50% would dramatically decrease the overall procedure time, allowing the physician to be more efficient and reducing the risk to the patient.

The example implementation of the actuator system was designed to reach a target in one DoF. The target can be reached after the trajectory is aligned to the DoF of the system. Modifications can be made to remotely control an adjusted frame in X and Y using the same type of hydrostatic actuators.

Conclusion:

A rolling diaphragm, hydrostatic actuator system is presented to assist with real-time MRI-guided minimally invasive interventions in targets with motion. This actuator system showed negligible effect on MR image artifact, SNR, and distortion. Its ability to transmit displacement from the master actuator to the slave actuator is demonstrated to have a linear response. Data from phantom experiments show that the actuator system can achieve targeted needle placement with significantly improved accuracy (mean NTE<about 2 mm) than the SS strategy. Using the actuator system, the insertion time of a targeting experiment can be reduced by about 50% and the total time reduced by about 25%. The actuator system can allow physicians to remotely perform real-time MM-guided interventions even while targets are in motion.

MR-Compatible Fluid Actuators for Robotic Interventions

Introduction:

A hydrostatic actuation system is presented that provides native haptic feedback, back-drivability, and MR-compatible transmission of force and displacement. Further, in the system, actuators can be connected to form a low-pressure, multi-master fluid network architecture to allow co-robotic operation—collaborative control of the end-effector by several master units—which can be fully human-controlled, fully robotic, or a combination. In this collaborative framework, the inputs of the various master units are blended in hardware, allowing a wide range of switchable modes of operation (e.g., master-slave, co-robotic, and fully autonomous) to be realized.

Co-robot (multi-master) operation involves multiple master units, under varying degrees of human and computer control, collaboratively controlling the output of a slave unit (e.g., a single slave unit). In some embodiments, the hydrostatic system is a closed system, so it can be seen that the net volumetric displacement of the master units will be the negative of the volumetric displacement of the slave unit; that is, the slave will sum the inputs of all of the connected masters. This allows for collaborative schemes, such as input scaling—where the robot sets its input such that the slave's output is a scaled version of a human's input, frequency crossover—where the human and robot each are responsible for some frequency band, and virtual wall—where the robot does not interfere with the human, until the slave unit is about to cross into a restricted zone, and then negates the human's input as long as the human attempts to move into that zone. Haptic feedback can be provided to users through the fluid pressure—shared across all units—or through electromechanical actuators attached to individual master units.

FIG. 22 gives an overview of a proposed closed-loop system. In this diagram, dark lines indicate signals, denoted lines indicate fluid transmission lines, and denoted lines indicate physical contact and manipulation. Because of a low data rate from the MR Image Processing module (e.g., implemented as processor-executable instructions), a data fusion problem is presented where the slave position is extrapolated from master units' encoders at a control loop rate, and is updated periodically by a lower-rate measurement from the MR Image Processing module.

Fluid Networks and Control:

Fluid Network Concepts.

To implement the co-robotic system, a fluid actuator network is developed—a variation on the master-slave configuration that features multiple master units. This architecture has many advantages, such as hardware blending of inputs, simultaneous haptic feedback capability between all units (e.g., each unit can receive actions of all others), and a streamlined method of incorporating more units. Unlike pneumatic or hydrodynamic systems, active components (e.g., pumps and valves) can be omitted, and operation of the system at high pressure can be omitted. The basic equation governing the operation of this fluid network is:

$${\sum }_{k = 0}^{n} {d}_{k} {A}_{k} + {d}_{s} {A}_{s} = 0$$

where dₖ denotes the linear displacement of each individual actuator k, dₛ the displacement of the slave actuator, and Aₖ the piston cross-sectional area of each actuator. When multiple master units are actuated in tandem, many modes of co-robotic operation can be implemented. For example, consider the case of one master unit controlled by a human (h) and sensed, one fully robotic master unit (r), and one slave unit (s). If the robotic unit follows the trajectory,

dᵣ=(1−α)dₕ

then the slave unit's output will be

dₛ=αdₕ

This technique can expand the precision available to beyond that which is usually attained in a manually-operated device by allowing the slave to perform fine manipulation from the human operator's gross manipulation.

Another mode of operation possible with the configuration above is a frequency crossover scheme, where the human unit is responsible for a certain frequency range of the desired signal, and the robot is responsible for the balance. This is implemented as

f_{a,h}=Qu(t), f_{a,r}=(a−Q)u(t)

where u(t) is the desired total control input, and Q is a filter. This can be used to reduce hand tremors during manual operation, to task the robot with tracking of a moving target while still allowing the physician manual course correction, or to add a high-frequency vibration to the slave actuator's motion to aid in tissue penetration.

Another mode of operation possible with this configuration is the virtual wall, where the human has full control until the output of the slave unit nears a restricted area (e.g., in a clinical application, an organ or bone). When this occurs, the robot engages, negating any further human inputs that would drive the slave unit into the restricted area, effectively nulling the slave unit's motion.

These three cases can be generalized to support more than two master units and arbitrary input strategies as follows:

$${f}_{a , k} = {Q}_{k} u ( t ) , {\sum }_{k}^{ } {Q}_{k} = 1$$

where each Qₖ can be a constant, filter, or nonlinear function, as long as the individual Qₖ sum to unity across the frequency band. Further, the filters Qₖ can be dynamically adjusted during operation to alter the blending scheme, or to switch certain actuators on or off in software. This allows a software-configurable, modular system that can switch from open-loop, human control to fully autonomous closed-loop target tracking (or somewhere in between), without hardware modification.

To achieve universal haptic feedback across all units, actuators should have low friction and stiction, lest the haptic force be drowned out by frictional forces. Further, to use the above basic equation to estimate the position of the slave unit—which would be beneficial given the low frame rate of the MR imagery—the system should be stiff, so system compliance contributes little to error in estimating the slave's position.

Fluid Network Demonstration.

To demonstrate the various modes of operation of the fluid network design, a one degree-of-freedom test bed is constructed. This setup includes three actuators: two master units, one human-controlled unit (“Human Master”) whose position is sensed, and a robot-controlled unit (“Robot Master”) whose position is sensed and controlled through an inner loop; and a single slave unit whose position is sensed. The three basic blending schemes discussed above are demonstrated.

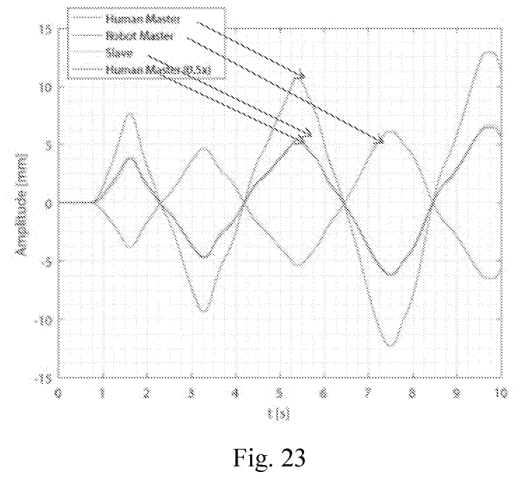

FIG. 23 shows the system in Motion Scaling mode. The positions of the three actuators are plotted, and a fourth virtual trace showing the theoretical motion of the slave unit is provided for reference. The Human Master was actuated by hand to simulate a human clinician, and it can be seen that the system accurately scales the human-controlled unit's motion. These results are from open-loop operation—the scaling is performed by the fluid network, involving sensing of just the master units' positions. The scale factor shown here was chosen arbitrarily, and though the system is capable of changing scale factor on-the-fly, this was not performed for clarity of presentation.

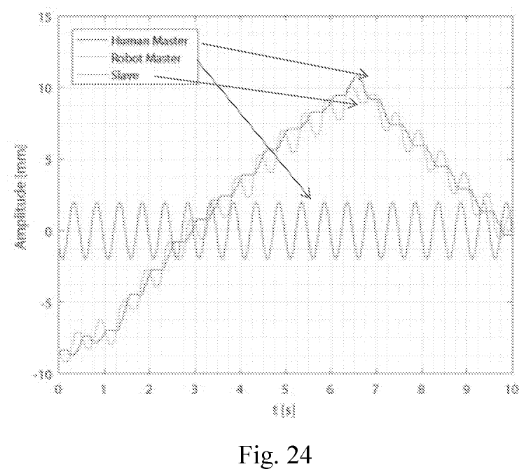

The second mode of operation, Frequency Crossover, is demonstrated in FIG. 24. In this experiment, an about 2-Hz dither signal was added to the output by the Robot Master, on top of manual actuation of the Human Master unit. The unevenness of the Human Master input is due to the effect of the system haptics from the Robot Master on the operator of the Human Master unit. As in the previous experiment, this was performed fully open-loop, with the slave output sensed strictly for verification purposes.

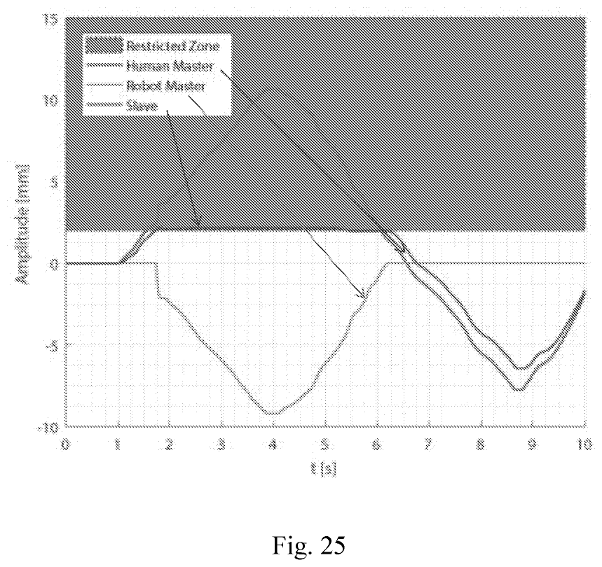

The final collaborative mode of operation to be demonstrated is the Virtual Wall. In the experiment shown in FIG. 25, the restricted zone was set to be the region where output is greater than 2 mm. For this experiment, sensing of the slave unit's position was performed, though the signal was not used for feedback. This strategy reduces excursions of the slave unit into the restricted zone by nullifying all human master inputs that would drive the slave unit into the restricted zone by actuating the robot master equally and opposite.

Fully-Autonomous Robotic Operation.

In addition to co-robotic operation, fully-autonomous target tracking is also a desirable mode of operation. Using real-time image feedback (performed by the MR Image Processing module), the system is able to provide direct slave unit position feedback using MR images. This provides closed-loop control using this image-based feedback to autonomously drive a needle to a pre-identified target in the presence of respiratory motion.

The system included three major subsystems: the Motion Phantom, the Robotic Manipulator, and the Scanner and image-processing infrastructure. A gelatin phantom is mounted on the Motion Phantom moving stage as it tracks a pre-recorded respiratory motion profile recorded from a live volunteer; this is the abdominal lesion analogue. The Robotic Manipulator is the robotic system described herein, with the Human Master input disabled and an MR-compatible biopsy needle attached to its output. The controller is implemented on a National Instruments PXI real-time target, which receives feedback data from the master-side encoders and from the Image Processing module through a serial link. For a target-tracking application, both a target position (reference) and needle position (output) can be obtained from the Image Processing module. Finally, the Scanner is a 3T MRI system (MAGNETOM Prisma, Siemens, Erlangen, Germany), connected through a custom imaging pipeline to the Image Processing module.

Controller Design

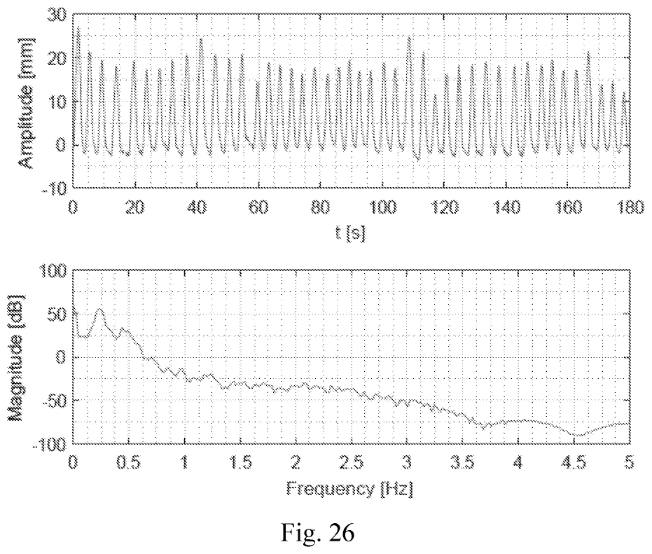

An example of typical breathing motion is given in FIG. 26. This data was recorded from a live human subject, and will serve as the reference trajectory for the tracking experiment. This trajectory is roughly periodic, though the period is unknown a priori and varies breath-to-breath. Additionally, it does not have a repeatable shape that can be taken advantage of for prediction, even if the period were constant. The non-stationary reference indicates that an adaptive controller with feedforward and feedback channels is desired.

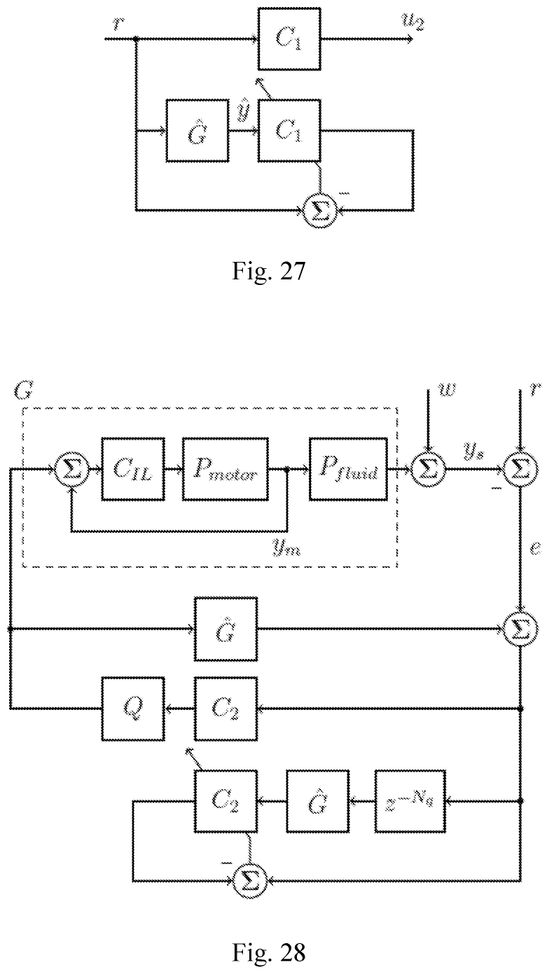

The feedforward channel of the controller is based on Widrow's adaptive inverse controller, and a block diagram is given in FIG. 27. G hat is the model of the pre-stabilized closed-loop plant G, C₂ is a finite impulse response (FIR) filter whose tap weights are determined by the Recursive Least Squares (RLS) procedure to minimize the error in inverting the closed-loop model—model matching problem to minimize

∥(1−FĜ)r∥₂.

As the adaptive controller is inverting the model of the closed-loop plant, rather than the plant itself, tracking performance can be determined by the accuracy of the model. To improve the performance when an exact model is unavailable, an adaptive feedback structure can be introduced.

The structure of the feedback controller is given in FIG. 28. C₂ is a FIR filter, with filter weights determined using the RLS procedure to minimize

∥(1−z^{-N}^{_{q}}C₂Ĝ)x∥₂,x=e+xC₂QĜ.

Q is a linear-phase low-pass filter, of order N_{q}.

In a formulation of this controller (Q=1; N_{q}=0), if it is assumed G hat→G, the objective function can be rewritten

$${min}_{{C}_{1}} {‖ {( 1 - {C}_{2} \hat{G} )}^{- 1} ( 1 - {C}_{2} \hat{G} ) e ‖}_{2} = {min}_{{C}_{1}} {‖ e ‖}_{2}$$

so that—provided the modeling error is small—the feedback controller is directly minimizing tracking error. However, in the formulation used here, the Q filter has a tunable parameter that allows trading performance for robustness.

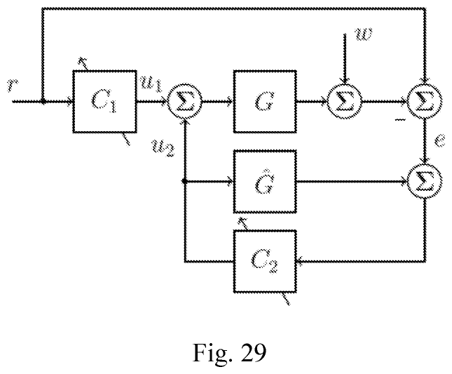

When these two channels are combined into a single controller, the structure of FIG. 29 is created. The transfer function from reference (r), and disturbance (w) to the tracking error (e) can be shown to be

$$e = \frac{( 1 - {C}_{2} \hat{G} ) ( 1 - {C}_{1} G )}{1 + {C}_{2} ( G - \hat{G} )} r - \frac{1 - {C}_{2} \hat{G}}{1 + {C}_{2} ( G - \hat{G} )} w$$

From this, it can be seen that minimizing

(1−C₂Ĝ)

will minimize tracking error from both the modeled system dynamics and the disturbance, allowing the controller to track references and reject disturbances. Functional blocks shown in FIGS. 27-29 can be implemented in hardware, or as processor-executable instructions stored in a memory.

### Controller

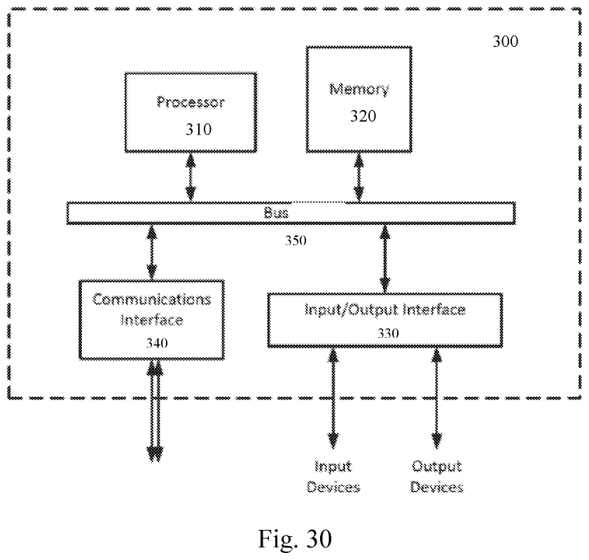

FIG. 30 shows an example of a controller 300 (or other computing device) that includes a processor 310, a memory 320, an input/output interface 330, and a communications interface 340. A bus 350 provides a communication path between two or more of the components of controller 300. The components shown are provided by way of example and are not limiting. The controller 300 may have additional or fewer components, or multiple of the same component.

The processor 310 represents one or more of a microprocessor, microcontroller, an application-specific integrated circuit (ASIC), and a field-programmable gate array (FPGA), along with associated logic.

The memory 320 represents one or both of volatile and non-volatile memory for storing information. Examples include semiconductor memory devices such as erasable programmable read-only memory (EPROM), electrically erasable programmable read-only memory (EEPROM), random-access memory (RAM), and flash memory devices, discs such as internal hard drives, removable hard drives, magneto-optical, compact disc (CD), digital versatile disc (DVD), and Blu-ray discs, memory sticks, and the like.

The functionality of the system of some embodiments can be implemented as processor-executable instructions in the memory 320, executed by the processor 310.

The input/output interface 330 represents electrical components and optional instructions that together provide an interface from the internal components of the controller 300 to external components. Examples include a driver integrated circuit with associated programming.

The communications interface 340 represents electrical components and optional instructions that together provide an interface from the internal components of the controller 300 to external networks.

The bus 350 represents one or more connections between components within the controller 300. For example, the bus 350 may include a dedicated connection between the processor 310 and memory 320 as well as a shared connection between the processor 310 and multiple other components of the controller 300.

### Example Embodiments

In an aspect according to some embodiments, a system for imparting motion of an object is provided. In some embodiments, the system includes at least one first hydrostatic actuator, a hydraulic transmission conduit, and at least one second hydrostatic actuator. The first hydrostatic actuator is connected (e.g., fluidly connected) to the second hydrostatic actuator via the hydraulic transmission conduit, such that an input displacement applied to the first hydrostatic actuator is transmitted via the hydraulic transmission conduit to the second hydrostatic actuator to impart motion of the object.

In some embodiments, the hydraulic transmission conduit has a first end and a second end, the first end is connected to the first hydrostatic actuator, and the second end is connected to the second hydrostatic actuator.

In some embodiments, the first hydrostatic actuator has a material composition that is primarily or entirely non-metallic. In some embodiments, the first hydrostatic actuator has a material composition that is primarily or entirely non-ferromagnetic. In some embodiments, the first hydrostatic actuator has a material composition that is primarily or entirely non-metallic and non-ferromagnetic. In some embodiments, the first hydrostatic actuator has a material composition that is primarily or entirely polymeric. In some embodiments, the first hydrostatic actuator is devoid of a ferromagnetic material. In some embodiments, the first hydrostatic actuator is devoid of a metal.

In some embodiments, the first hydrostatic actuator is a first rolling diaphragm actuator. In some embodiments, the first rolling diaphragm actuator includes an actuator body, a piston moveably disposed within the actuator body, and a diaphragm extending between a portion of the actuator body and an end of the piston. In some embodiments, the first rolling diaphragm actuator includes an actuator body, a dual piston moveably disposed within the actuator body, and a pair of diaphragms extending between respective portions of the actuator body and respective ends of the dual piston. In some embodiments, the actuator body defines at least one slot, and the first rolling diaphragm actuator further includes at least one extension member that extends (e.g., outwardly or inwardly) from the piston (or the dual piston) and through the slot of the actuator body.

In some embodiments, the second hydrostatic actuator has a material composition that is primarily or entirely non-metallic. In some embodiments, the second hydrostatic actuator has a material composition that is primarily or entirely non-ferromagnetic. In some embodiments, the second hydrostatic actuator has a material composition that is primarily or entirely non-metallic and non-ferromagnetic. In some embodiments, the second hydrostatic actuator has a material composition that is primarily or entirely polymeric. In some embodiments, the second hydrostatic actuator is devoid of a ferromagnetic material. In some embodiments, the second hydrostatic actuator is devoid of a metal.

In some embodiments, the second hydrostatic actuator is a second rolling diaphragm actuator. In some embodiments, the second rolling diaphragm actuator includes an actuator body, a piston moveably disposed within the actuator body, and a diaphragm extending between a portion of the actuator body and an end of the piston. In some embodiments, the second rolling diaphragm actuator includes an actuator body, a dual piston moveably disposed within the actuator body, and a pair of diaphragms extending between respective portions of the actuator body and respective ends of the dual piston. In some embodiments, the actuator body defines at least one slot, and the second rolling diaphragm actuator further includes at least one extension member that extends (e.g., outwardly or inwardly) from the piston (or the dual piston) and through the slot of the actuator body.

In some embodiments, the system further includes a motor connected to the first hydrostatic actuator to apply the input displacement to the first hydrostatic actuator. In some embodiments, the system further includes a controller connected to the motor to direct operation of the motor. In some embodiments, the controller is configured to direct operation of the motor to impart motion of the object corresponding to a specified (e.g., pre-recorded or pre-derived) motion trajectory. In some embodiments, the controller is configured to direct operation of the motor to impart an oscillatory motion of the object. In some embodiments, the controller is configured to direct operation of the motor to impart motion of the object corresponding to a pre-recorded respiratory motion trajectory. In some embodiments, the controller is configured to direct operation of the motor to impart motion of the object, according to a feedforward control scheme or an iterative learning control scheme. In some embodiments, the controller is configured to direct operation of the motor to impart motion of the object, according to a feedback control scheme. In some embodiments, the feedback control scheme is according to a measured position of the object, such as from a set of acquired MR images while the object is within an MR scanner bore.

In some embodiments, the system includes multiple first hydrostatic actuators, including the first hydrostatic actuator, such that input displacements applied to the first hydrostatic actuators are transmitted via the hydraulic transmission conduit to the second hydrostatic actuator to impart motion of the object. In some embodiments, the first hydrostatic actuators have a same size, or have different sizes. In some embodiments, each of the first hydrostatic actuators is configured to change fluid displacement and hence move the second hydrostatic actuator, via manual or motor control. In some embodiments, the system further includes multiple motors connected to respective ones of the first hydrostatic actuators to apply the input displacements to the first hydrostatic actuators. In some embodiments, the system further includes one or more motors connected to respective ones of a subset of the first hydrostatic actuators, and a remaining subset of the first hydrostatic actuators operate via manual control. In some embodiments, the controller is configured to direct operation of the motor (or the motors) to impart motion of the object corresponding to a specified (e.g., pre-recorded or pre-derived) motion trajectory.

In some embodiments, the system includes multiple second hydrostatic actuators, including the second hydrostatic actuator. In some embodiments, the system includes multiple first hydrostatic actuators, including the first hydrostatic actuator, and multiple second hydrostatic actuators, including the second hydrostatic actuator, to impart multiple degrees-of-freedom motion of the object. In some embodiments, the first hydrostatic actuators are connected to the second hydrostatic actuators via the same hydraulic transmission conduit, or via respective and different hydraulic transmission conduits.

In some embodiments, the system further includes a holder to accommodate the object, and the holder is connected to the second hydrostatic actuator. In some embodiments, the object is a needle. In some embodiments, the object is a catheter. In some embodiments, the object is a phantom. In some embodiments, the system further includes a moving stage to accommodate the phantom, and the moving stage is connected to the second hydrostatic actuator.

In another aspect according to some embodiments, a method of operating the system according to any of the foregoing embodiments is provided. In some embodiments, the method includes placing the second hydrostatic actuator within a Magnetic Resonance (MR) scanner bore, and imparting motion of the object, via the second hydrostatic actuator, while the object is within the MR scanner bore. In some embodiments, the method further includes acquiring a set of MR images while the object is within the MR scanner bore.

In a further aspect according to some embodiments, a motion emulation system is provided. In some embodiments, the system includes: (1) a first hydrostatic actuator, devoid of ferromagnetic materials, configured to generate a hydraulic signal, the hydraulic signal corresponding to pre-recorded motion data; (2) a hydraulic transmission conduit having a first end and a second end, the first end connected to the first hydrostatic actuator, the hydraulic transmission conduit configured to transfer the hydraulic signal received at the first end to a transferred hydraulic signal at the second end; and (3) a second hydrostatic actuator, devoid of ferromagnetic materials, connected to the second end of the hydraulic transmission conduit, configured to transform the transferred hydraulic signal into a corresponding motion of an object.

As used herein, the singular terms “a,” “an,” and “the” may include plural referents unless the context clearly dictates otherwise. Thus, for example, reference to an object may include multiple objects unless the context clearly dictates otherwise.

As used herein, the term “set” refers to a collection of one or more objects. Thus, for example, a set of objects can include a single object or multiple objects.

As used herein, the terms “connect,” “connected,” and “connection” refer to an operational coupling or linking. Connected objects can be directly coupled to one another or can be indirectly coupled to one another, such as via one or more other objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, concentrations, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a range of about 1 to about 200 should be understood to include the explicitly recited limits of about 1 and about 200, but also to include individual values such as about 2, about 3, and about 4, and sub-ranges such as about 10 to about 50, about 20 to about 100, and so forth.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

What is claimed is:

1. A system for imparting motion of an object, comprising:
at least one first hydrostatic actuator;
a hydraulic transmission conduit; and
at least one second hydrostatic actuator,
wherein the first hydrostatic actuator is connected to the second hydrostatic actuator via the hydraulic transmission conduit, such that an input displacement applied to the first hydrostatic actuator is configured to be transmitted via the hydraulic transmission conduit to the second hydrostatic actuator to impart motion of the object.

2. The system of claim 1, wherein at least one of the first hydrostatic actuator or the second hydrostatic actuator has a material composition that is entirely polymeric.

3. The system of claim 1, wherein at least one of the first hydrostatic actuator or the second hydrostatic actuator is devoid of a metal.

4. The system of claim 1, wherein at least one of the first hydrostatic actuator or the second hydrostatic actuator is devoid of a ferromagnetic material.

5. The system of claim 1, wherein the first hydrostatic actuator is a first rolling diaphragm actuator.

6. The system of claim 5, wherein the first rolling diaphragm actuator includes an actuator body, a piston moveably disposed within the actuator body, and a pair of diaphragms extending between respective portions of the actuator body and respective ends of the piston.

7. The system of claim 6, wherein the actuator body defines a slot, and the first rolling diaphragm actuator further includes an extension member that extends from the piston and through the slot of the actuator body.

8. The system of claim 5, wherein the second hydrostatic actuator is a second rolling diaphragm actuator.

9. The system of claim 8, wherein the second rolling diaphragm actuator includes an actuator body, a piston moveably disposed within the actuator body, and a pair of diaphragms extending between respective portions of the actuator body and respective ends of the piston.

10. The system of claim 9, wherein the actuator body defines a slot, and the second rolling diaphragm actuator further includes an extension member that extends from the piston and through the slot of the actuator body.

11. The system of claim 10, further comprising a holder to accommodate the object, and the holder is connected to the extension member of the second rolling diaphragm actuator.

12. The system of claim 1, further comprising a motor connected to the first hydrostatic actuator to apply the input displacement to the first hydrostatic actuator.

13. The system of claim 12, further comprising a controller connected to the motor to direct operation of the motor.

14. The system of claim 13, wherein the controller is configured to direct operation of the motor to impart motion of the object corresponding to a specified motion trajectory.

15. The system of claim 14, wherein the controller is configured to direct operation of the motor to impart motion of the object, according to a feedforward control scheme or an iterative learning control scheme.

16. The system of claim 14, wherein the controller is configured to direct operation of the motor to impart motion of the object, according to a feedback control scheme.

17. The system of claim 16, wherein the feedback control scheme is according to a position of the object.

18. The system of claim 1, further comprising a plurality of first hydrostatic actuators, including the first hydrostatic actuator, connected to the second hydrostatic actuator via the hydraulic transmission conduit, such that input displacements applied to the first hydrostatic actuators are configured to be transmitted via the hydraulic transmission conduit to the second hydrostatic actuator to impart motion of the object.

19. The system of claim 18, further comprising a plurality of motors connected to respective ones of the first hydrostatic actuators.

20. The system of claim 18, further comprising at least one motor connected to at least one of the first hydrostatic actuators.

21. The system of claim 1, further comprising a plurality of first hydrostatic actuators, including the first hydrostatic actuator, and a plurality of second hydrostatic actuators, including the second hydrostatic actuator, to impart multiple degrees-of-freedom motion of the object.

22. A method of operating the system of claim 1, comprising:
placing the second hydrostatic actuator within a Magnetic Resonance (MR) scanner bore; and
imparting motion of the object, via the second hydrostatic actuator, while the object is within the MR scanner bore.

23. The method of claim 22, further comprising acquiring a set of MR images while the object is within the MR scanner bore.

24. The method of claim 22, wherein the object is a needle, a catheter, or a phantom.
